(19) United States
(12) Patent Application Publication
(10) Pub. No.: US 2025/0243176 A1
(43) Pub. Date: Jul. 31, 2025

(54) (S)-1-(1-ACRYLOYLPIPERIDIN-3-YL)-2-FLUORO-5,6,7,8,9,10-HEXAHYDROCYCLO HEPTA[B]INDOLE-4-CARBOXAMIDE, AND RELATED CRYSTALLINE FORMS, COMPOSITIONS, AND METHODS THEREOF

(71) Applicant: GB005, Inc., San Diego, CA (US)

(72) Inventors: Beili ZHANG, San Diego, CA (US); Fahmi AL-AQRABI, San Diego, CA (US)

(21) Appl. No.: 18/702,652

(22) PCT Filed: Oct. 18, 2022
(86) PCT No.: PCT/US2022/047050

§ 371 Date: Apr. 18, 2024

Related U.S. Application Data
(60) Provisional application No. 63/393,163, filed on Jul. 28, 2022.
(60) Provisional application No. 63/257,509, filed on Oct. 19, 2021.

(51) Int. Cl. C07D 401/04; A61K 9/00; A61K 31/454; A61K 45/06; A61K 47/10; A61K 47/22

(52) U.S. Cl. CPC C07D 401/04; A61K 31/454; A61K 45/06; A61K 47/10; A61K 47/22; A61K 9/0053

(57) ABSTRACT

Crystalline forms of (5)-1-(1-acryloylpiperidn-3-yl)-2-fluoro-5,6,7,8,9,10-hexahydrocyclo hepta[b]indole-4-carboxamide are provided. Pharmaceutical compositions containing (S)-1-(1-acryloylpiperidin-3-yl)-2-fluoro-5,6,7,8,9,10-hexahydrocyc lohepta[b]indole-4-carboxamide are also provided, as well as related methods for their preparation and use in modulating kinases generally, and specifically to treatment of cancer.

### FIELD OF THE INVENTION

The present invention relates to crystalline forms of (S)-1-(1-acryloylpiperidin-3-yl)-2-fluoro-5,6,7,8,9,10-hexahydrocyclohepta[b]indole-4-carboxamide, as well as to products comprising (S)-1-(1-acryloylpiperidin-3-yl)-2-fluoro-5,6,7,8,9,10-hexahydrocyclohepta[b]indole-4-carboxamide, and related methods of their use and preparation.

### BACKGROUND

Protein kinases are a large group of intracellular and transmembrane signaling proteins in eukaryotic cells. These enzymes are responsible for transfer of the terminal (gamma) phosphate from ATP to specific amino acid residues of target proteins. Phosphorylation of specific amino acid residues in target proteins can modulate their activity leading to profound changes in cellular signaling and metabolism. Protein kinases can be found in the cell membrane, cytosol and organelles such as the nucleus and are responsible for mediating multiple cellular functions including metabolism, cellular growth and differentiation, cellular signaling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: TEC, SRC, ABL, JAK, CSK, FAK, SYK, FER, ACK and the receptor tyrosine kinase subfamilies including ERBB, FGFR, VEGFR, RET and EPH. Subclass I of the receptor tyrosine kinase superfamily includes the ERBB receptors and comprises four members: ErbB1 (also called epidermal growth factor receptor (EGFR)), ErbB2, ErbB3 and ErbB4.

Kinases exert control on key biological processes related to health and disease. Furthermore, aberrant activation or excessive expression of various protein kinases are implicated in the mechanism of multiple diseases and disorders characterized by benign and malignant proliferation, as well as diseases resulting from inappropriate activation of the immune system. Thus, inhibitors of select kinases or kinase families are considered useful in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, ischemia, dermatomyositis, pemphigus, and the like.

Tec kinases are a family of non-receptor tyrosine kinases predominantly, but not exclusively, expressed in cells of hematopoietic origin. The Tec family includes TEC, Bruton's tyrosine kinase (BTK), inducible T-cell kinase (ITK), resting lymphocyte kinase (RLK/TXK for Tyrosine Protein Kinase), and bone marrow-expressed kinase (BMX/ETK).

BTK is important in B-cell receptor signaling and regulation of B-cell development and activation. Mutation of the gene encoding BTK in humans leads to X-linked agammaglobulinemia which is characterized by reduced immune function, including impaired maturation of B-cells, decreased levels of immunoglobulin and peripheral B cells, and diminished T-cell independent immune response. BTK is activated by Src-family kinases and phosphorylates PLC gamma leading to effects on B-cell function and survival. Additionally, BTK is important for cellular function of mast cells, macrophage and neutrophils indicating that BTK inhibition is effective in treatment of diseases mediated by these and related cells including inflammation, bone disorders, and allergic disease. BTK inhibition is also important in survival of lymphoma cells indicating that inhibition of BTK is useful in the treatment of lymphomas and other cancers. As such, inhibitors of BTK and related kinases are of great interest as anti-inflammatory, as well as anti-cancer, agents. BTK is also important for platelet function and thrombus formation indicating that BTK-selective inhibitors are also useful as antithrombotic agents. Furthermore, BTK is required for inflammasome activation, and inhibition of BTK may be used in treatment of inflammasome-related disorders, including stroke, gout, type 2 diabetes, obesity-induced insulin resistance, atherosclerosis and Muckle-Wells syndrome. In addition, BTK is expressed in HIV infected T-cells and treatment with BTK inhibitors sensitizes infected cells to apoptotic death and results in decreased virus production. Accordingly, BTK inhibitors are considered useful in the treatment of HIV-AIDS and other viral infections.

(S)-1-(1-acryloylpiperidin-3-yl)-2-fluoro-5,6,7,8,9,10-hexahydrocyclohepta[b]indole-4-carboxamide, hereinafter referred to as “Compound 1”, is an orally available, selective, potent inhibitor of Bruton's tyrosine kinase (BTK), thereby providing potential treatment options in BTK-driven diseases. The potency of Compound 1 for BTK inhibition is in the nM range in both cell-free enzymatic and whole blood functional assays. It is a central nervous system (CNS) penetrant and demonstrates rapid BTK inactivation kinetics, in both peripheral and CNS tissue. In a kinome scan, Compound 1 exhibits high kinase selectivity against 349 kinases with only two kinases (TEC and TXK) demonstrating >50% inhibition at 1 μM.

An amorphous form of Compound 1 (i.e., Compound 5-6) has been described in U.S. patent application Ser. No. 17/225,984, which published as U.S. Patent Application Publication No. US 2022/0009920 (incorporated herein by reference in its entirety). Compound 1 has the chemical formula C₂₂H₂₆FN₃O₂, a molecular weight of 383.47, and the following structure:

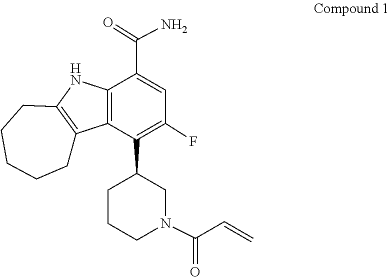 [SMILES: C=CC(=O)N1CCC[C@@H](c2c(F)cc(C(N)=O)c3[nH]c4c(c23)CCCCC4)C1]

Given the clinical promise of Compound 1, there is a need for new, improved and/or enhanced forms of Compound 1, particularly in the context of pharmaceutical drug products suitable for oral administration, as well as for compositions comprising Compound 1 and methods related to the manufacture and use of the same. The present invention fulfils these and related needs, as evidenced by the following detailed description and attached drawings.

### BRIEF SUMMARY

Solids drug forms may exist in either amorphous or crystalline states. In the case of crystalline forms, molecules are positioned in 3-dimensional lattice sites. When a compound recrystallizes from a solution or slurry, it may crystallize with different spatial lattice arrangements, a property referred to as “polymorphism,” with the different crystal forms being referred to as “polymorphs” or individually as a “polymorph”. Different polymorphs of a given substance may differ from each other with respect to one or more physical properties, such as solubility and dissolution, true density, crystal shape, compaction behavior, flow properties, and/or solid state stability. In the case of a chemical substance that exists in two (or more) polymorphic forms, unstable form(s) generally convert to the more thermodynamically stable form(s) at a given temperature after a sufficient period of time. When this transformation is not rapid, the thermodynamically unstable form is referred to as the “metastable” form. In general, the stable form exhibits the lowest solubility, and the maximum chemical stability. However, the metastable form may exhibit sufficient chemical and physical stability under normal storage conditions to permit its use in a commercial form. In this case, the metastable form, although less stable, may exhibit properties desirable over those of the stable form, such as enhanced solubility or better oral bioavailability.

Accordingly, in one embodiment, novel solid crystalline forms of Compound 1 are provided. In more specific embodiments, the novel solid crystalline forms are six different polymorphs of Compound 1, which are referred to herein as “Form I”, “Form II”, “Form III”, “Form IV”, “Form V”, “Form VI”, and “Form VII”.

In an embodiment, a crystalline form of Compound 1 is provided wherein the crystalline form is Form I, and in a further embodiment is substantially pure Form I. Form I may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form II, and in a further embodiment is substantially pure Form II. Form II may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form III, and in a further embodiment is substantially pure Form III. Form III may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form IV, and in a further embodiment is substantially pure Form IV. Form IV may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form V, and in a further embodiment is substantially pure Form V. Form V may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form VI, and in a further embodiment is substantially pure Form VI. Form VI may be characterized by the various analytical techniques disclosed herein, including (for example) by X-ray powder diffraction (XRPD) and the characteristic diffractograms generated by the same.

In an embodiment, a crystalline form of Compound 1 is provided wherein the crystalline form is Form VII, and in a further embodiment is substantially pure Form VII. Form VII may be characterized by the various analytical techniques disclosed herein, including (for example) by X-ray powder diffraction (XRPD) and the characteristic diffractograms generated by the same.

In other embodiments, a crystalline form of Compound 1 is provided wherein the crystalline form is a mixture of two or more Forms. As defined below, a mixture is provided when one crystalline form is present at a ratio ranging from of 5-95% by weight of the other crystalline form or forms (ratios above or below this range are characteristic of substantially pure crystalline forms).

In other embodiments, processes are provided for preparing the solid crystalline forms of Compound 1.

In other embodiments, a pharmaceutical composition is provided comprising Compound 1 in combination with one or more pharmaceutically acceptable carriers. Such compositions may be formulated in a variety for different forms. For example, the composition may be formulated for oral administration.

In an embodiment, the pharmaceutical composition may comprise an additional therapeutically active agent (i.e., in addition to Compound 1), or such additional therapeutically active agent may be present as a separate pharmaceutical composition and co-administered with Compound 1 (e.g., at the same time).

In a further embodiment, the additional therapeutically active agent is a corticosteroid, a noncorticosteroidal, an immunosupressive and/or an antiinflammatory agent. In a more specific embodiment, the immunosuppressive agent is selected from interferon alpha, interferon gamma, cyclophosphamide, tacrolimus, mycophenolate mofetil, methotrexate, dapsone, sulfasalazine, azathioprine, an anti-CD20 agent (such as rituximab, ofatumumab, obinutuzumab, or veltuzumab, or a biosimilar version thereof), an anti-TNFalpha agent (such as entanercept, infliximab, golilumab, adalimumab, or certolizumab pegol or a biosimilar version thereof), an anti-IL6 agent toward ligand or its receptors (such as tocilizumab, sarilumab, olokizumab, elsililumab, or siltuximab), an anti-IL17 agent to ligand or its receptors (such as secukinumab, ustekinumab, brodalumab, or ixekizumab), an anti-L1 agent to ligand or its receptors (such as with rilonacept, canakinumab, or anakinra), an anti-IL2 agent to ligand or its receptors (such as basiliximab or daclizumab), an anti-CD2 agent such as alefacept, an anti-CD3 agent such as muromonab-cd3, an anti-CD80/86 agent such as abatacept or belatacept, an anti-sphingosine-1-phosphate receptor agent such as fingolimod, an anti-C5 agent such as eculizumab, an anti-integrin alpha4 agent such as natalizumab, an anti-α₄β₇ agent such as vedolizumab, an anti-mTOR agent such as sirolimus or everolimus, an anti-calcineurin agent such as tacrolimus, an anti-BAFF/BlyS agent (such as belimumab, VAY736, or blisibimod), leflunomide and teriflunomide.

In a further embodiment, the additional therapeutically active agent is an immunosuppressive agent, such as rituximab, ofatumumab, obinutuzumab, veltuzumab, or a biosimilar version thereof.

In a further embodiment, the additional therapeutically active agent is an immunomodulator imide drug (IMiD) such as thalidomide and its analogues (lenalidomide, pomalidomide and iberdomide), a checkpoint blockade agent such as anti-PD1, anti-CTLA4, anti-Tim3 and ant-Lag3 monoclonal antibodies, an anti-CD19 monoclonal antibody such as inebilizumab and tafasitamab, an IRAK inhibitor, a chemotherapy agent such as methotrexate and temozolomide, or anti-CD19 CAR T cell therapy.

In other embodiments, the pharmaceutical composition comprises polyethylene glycol. In another embodiment the pharmaceutical composition comprises polyethylene glycol and/or propylene glycol monolaurate. In other embodiments, the pharmaceutical composition comprises vitamin E. In other embodiments, the pharmaceutical composition comprises butylated hydroxytoluene (BHT). In some embodiments, the pharmaceutical composition comprises 1-25 mg of Compound 1.

In another embodiment, a method is provided for treating a disease or condition modulated by kinase inhibition, comprising administering to a subject in need thereof an effective amount of Compound 1, or a pharmaceutical composition comprising the same. In a more specific embodiment, the kinase is a tyrosine kinase such as (but not limited to) BTK.

In an embodiment, the disease or condition is cancer, an autoimmune disease, an inflammatory disease, or a thromboembolic disease.

In one embodiment, a use of Compound 1 or a crystalline form of Compound 1, or a pharmaceutical composition thereof, is provided, in the manufacture of a medicament.

### BRIEF DESCRIPTION OF THE DRAWINGS

FIG. 1: Form I XRPD pattern.

FIG. 2: Form I TGA and DSC thermograms.

FIG. 3: Form II XRPD pattern.

FIG. 4: Form II TGA and DSC thermograms.

FIG. 5: Form III XRPD pattern.

FIG. 6: Form III TGA and DSC thermograms.

FIG. 7: Form III DVS plot.

FIG. 8: Form III XRPD patterns before and after DVS testing.

FIG. 9: Form IV XRPD patterns.

FIG. 10: Form IV TGA and DSC thermograms.

FIG. 11: Form V XRPD patterns heated to 85° C. and 155° C.

FIG. 12: Form V TGA and DSC thermograms.

FIG. 13: Form VI XRPD pattern.

FIG. 14: Form VI TGA and DSC thermograms.

FIG. 15: Form VII XRPD pattern.

FIG. 16: Form VII DSC thermogram.

FIG. 17: Form VII TGA thermogram.

FIG. 18: Overlay of XRPD patterns of Forms I-VII.

FIG. 19: Proposed crystal form conversion.

FIG. 20: Form III DSC thermogram.

FIG. 21: Form III TGA and DSC thermograms.

FIG. 22: Form III XRPD patterns−sStability analysis.

FIG. 23: Form III single crystal X-ray structure.

FIG. 24: Manufacturing process overview.

FIG. 25: Plasma profile Mean±SD.

FIG. 26: CSF to unbound plasma ratio by dose.

FIG. 27: Single and multiple dose plasma profile (Mean±SD).

FIG. 28: 15 mg fasted, moderate-fat meal, and high-fat meal plasma profiles (Mean±SD).

FIG. 29: Plasma profile of Compound 1 alone and with Itraconazole (Mean±SD).

### DETAILED DESCRIPTION

According to the present disclosure, novel solid crystalline forms of Compound 1 are provided. In more specific embodiments, the novel solid crystalline forms are seven different polymorphs of Compound 1; namely, Form I, Form II, Form III, Form IV, Form V, Form VI or Form VII. These forms differ from the amorphous form of Compound 1 in the structure of the crystal lattice, with each form giving distinctive x-ray powder diffraction (XRPD) patterns and differential scanning calorimeter (DSC) thermograms.

As used herein “amorphous” refers to a lack of well-ordered diffraction lines resulting from the absence of a repeated crystal lattice.

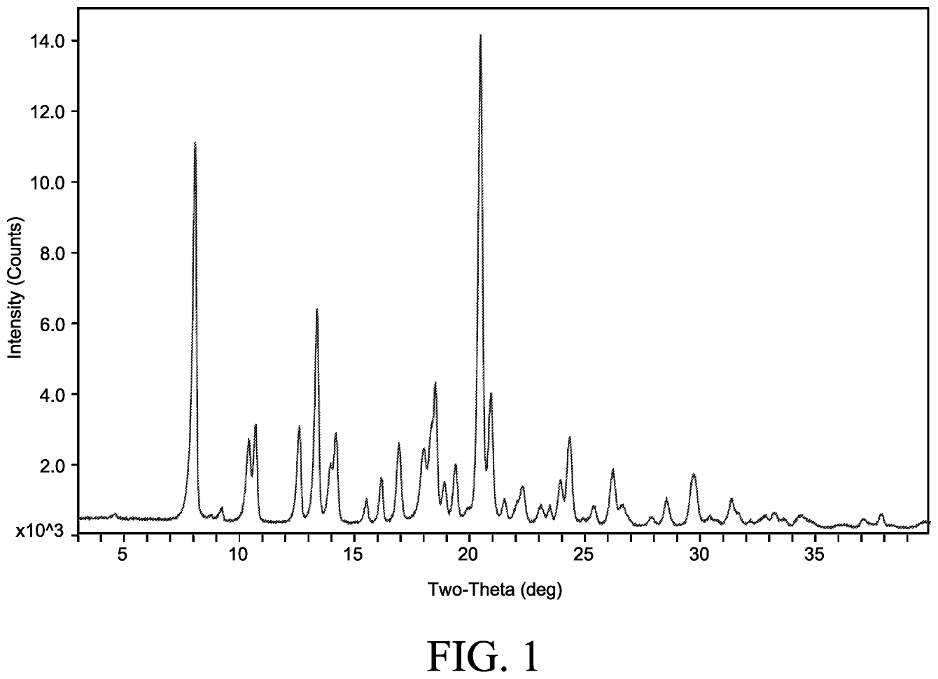

In one embodiment the present disclosure provides Form I, characterized by a XRPD pattern having peaks at 9.2011, 13.9620 and 16.1506±0.2 degrees 2-theta. In another embodiment Form I is provided further characterized by an XRPD pattern substantially as shown in FIG. 1.

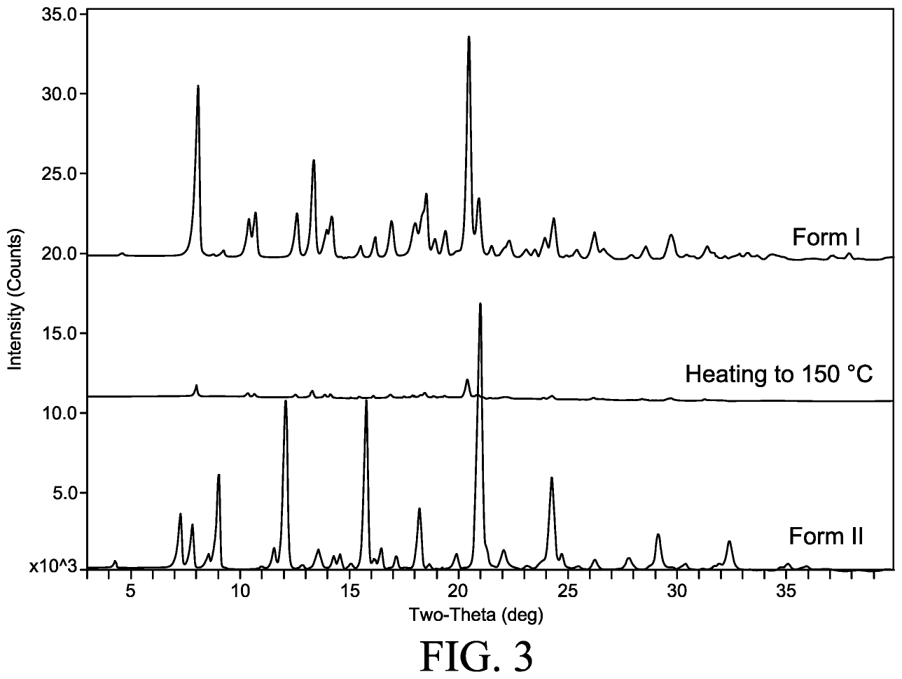

In one embodiment the present disclosure provides Form II, characterized by a XRPD pattern having peaks at 4.2759, 8.5794 and 24.2411±0.2 degrees 2-theta. In another embodiment Form II is provided further characterized by an XRPD pattern substantially as shown in FIG. 3.

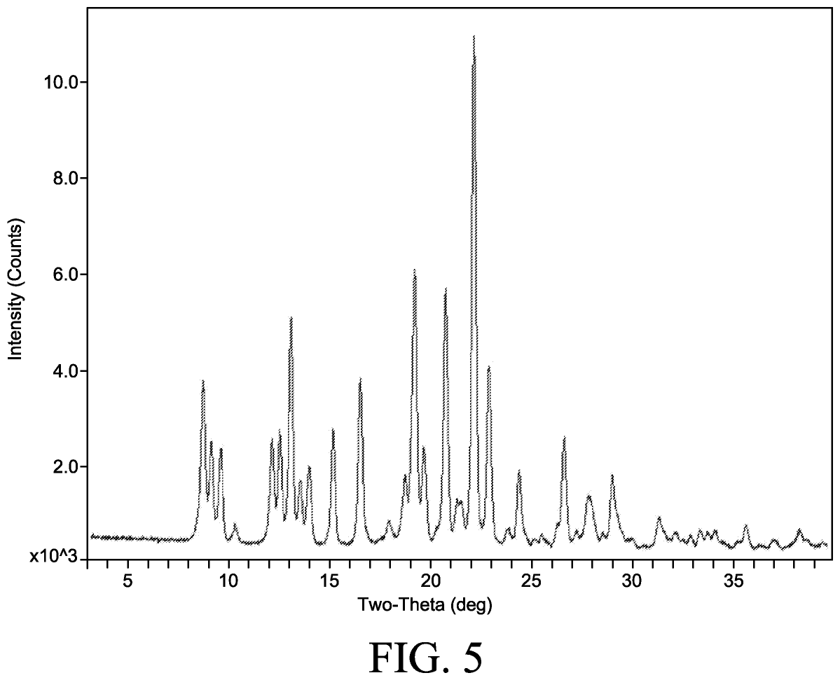

In one embodiment the present disclosure provides Form III, characterized by a XRPD pattern having peaks at 10.2543, 13.5006 and 13.9691±0.2 degrees 2-theta. In another embodiment Form III is provided further characterized by an XRPD pattern substantially as shown in FIG. 5.

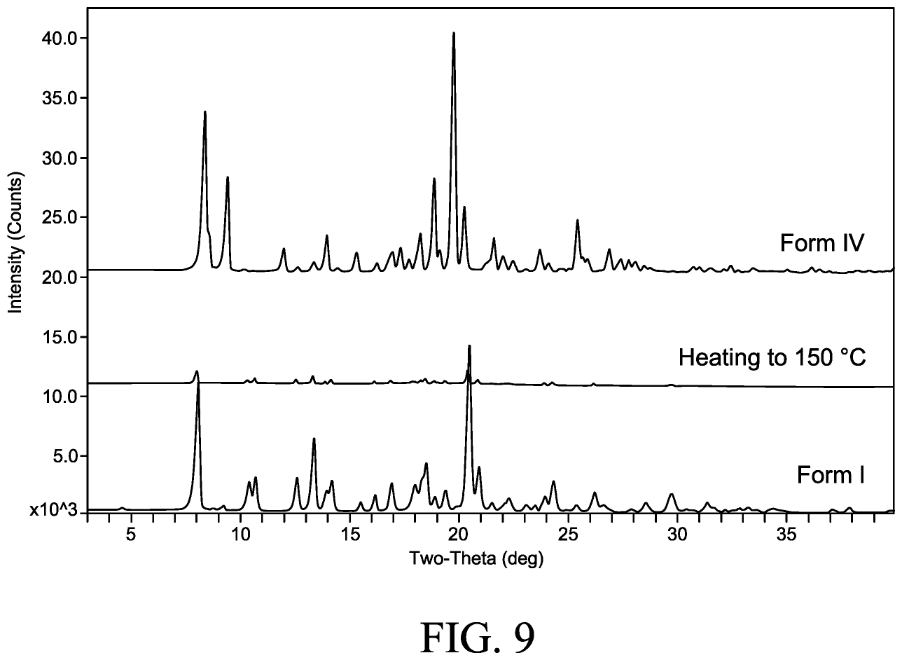

In one embodiment the present disclosure provides Form IV, characterized by a XRPD pattern having peaks at 8.6027, 11.9598, 13.9360, 21.5845 and 25.4090±0.2 degrees 2-theta. In another embodiment Form IV is provided further characterized by an XRPD pattern substantially as shown in FIG. 9.

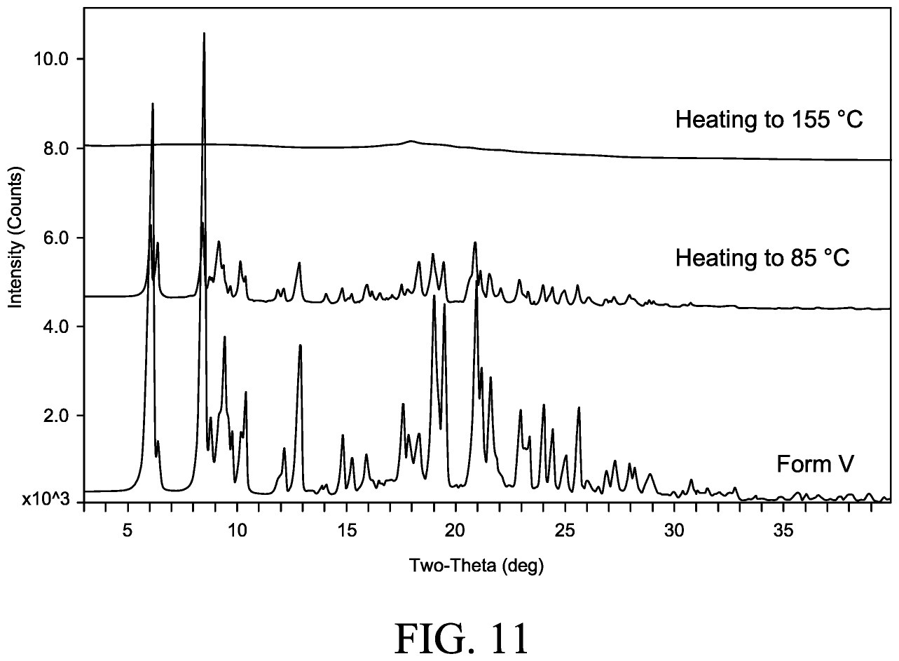

In one embodiment the present disclosure provides Form V, characterized by a XRPD pattern having peaks at 6.4014, 9.1908, 14.8143, 17.5539, 21.5891, 23.9883 and 25.5807±0.2 degrees 2-theta. In another embodiment Form V is provided further characterized by an XRPD pattern substantially as shown in FIG. 11.

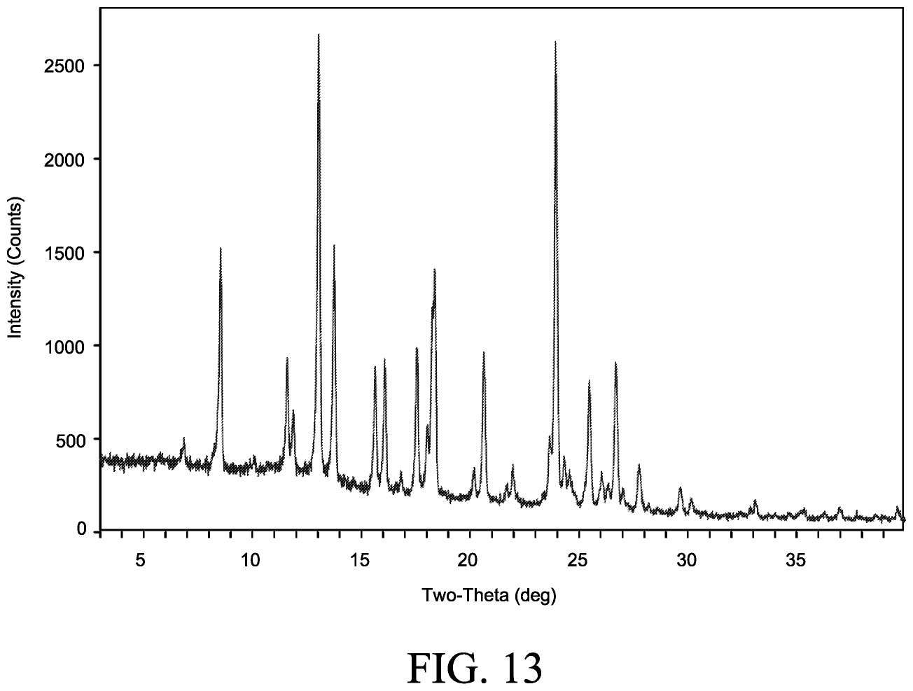

In one embodiment the present disclosure provides Form VI, characterized by a XRPD pattern having peaks at 6.8339, 10.1404, 15.6784, 16.1217, 17.5940, 20.6765, 25.5122 and 26.7363±0.2 degrees 2-theta. In another embodiment Form VI is provided further characterized by an XRPD pattern substantially as shown in FIG. 13.

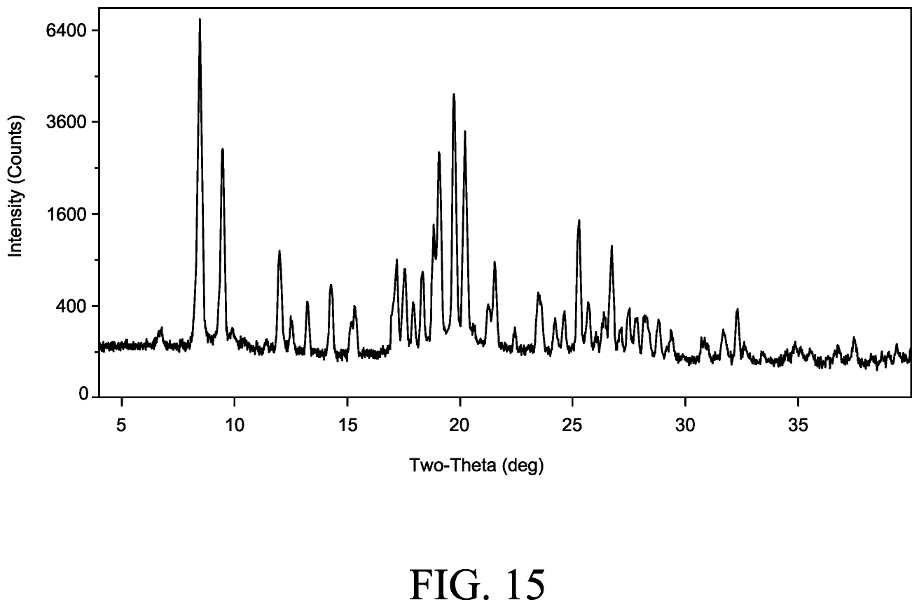

In one embodiment the present disclosure provides Form VII, characterized by a XRPD pattern having peaks at 6.727, 8.4799, 9.4854, 12.0161, 17.1901, 18.8407, 19.0691, 19.7285 and 20.2268±0.2 degrees 2-theta. In another embodiment Form VII is provided further characterized by an XRPD pattern substantially as shown in FIG. 15.

In the practice of this invention, a single polymorph (i.e., Form I, Form II, Form III, Form IV, Form V, Form VI or Form VII) may be utilized in a substantially pure form, or may be utilized as a mixture of one or more polymorphs.

In a further embodiment, a method is provided for treating a disease or condition modulated by kinase inhibition, comprising administering to a subject in need thereof an effective amount of a solid crystalline form of Compound 1; namely, Form I, Form II, Form III, Form IV, Form V, Form VI or Form VII.

In one embodiment, the kinase is a tyrosine kinase, and in a more specific embodiment is Bruton's tyrosine kinase (BTK).

In one embodiment, the disease or condition modulated by kinase inhibition is cancer.

The present invention is further illustrated by the following examples, which should not be construed as limiting in any way.

### EXAMPLES

### Polymorph Screening

Polymorph screening of Compound 1 was conducted using various crystallization methods, including slurry, cooling and evaporative crystallization, anti-solvent precipitation, thermal and mechanical treatment.

### Example 1: Analytical Methods

### Example 1A: X-Ray Powder Diffraction (XRPD)

XRPD patterns were identified with an X-ray diffractometer (PANalytical Empyrean). The system was equipped with PIXcel^{ID} detector. Samples were scanned from 3 to 40° 2θ, at a step size of 0.013° 2θ. The tube voltage and current were 45 KV and 40 mA, respectively. (Form VII was scanned from 4 to 40° 2θ, at a step size of 0.011° 2θ, the tube voltage and current were 40 KV and 15 mA, respectively.)

### Example 1B: Differential Scanning Calorimeter (DSC)

DSC was performed using a Discovery DSC 250 (TA Instruments, US) (for Form VII, Discovery DSC Q2000 was used). The sample was placed into an aluminum pin-hole hermetic pan and the weight was accurately recorded. Then the sample was heated at a rate of 10° C./min from 25° C. to the final temperature.

### Example 1C: Thermogravimetric Analysis (TGA)

TGA was carried out on a Discovery TGA 55 (TA Instruments, US) (for Form VII, Discovery TGA Q500 was used). The sample was placed into an open tared aluminum pan, automatically weighed, and inserted into the TGA furnace. The sample was heated at a rate of 10° C./min from room temperature (RT) to the final temperature.

### Example 1D: Dynamic Vapor Sorption (DVS)

Moisture sorption/desorption data was collected on a Vsorp Dynamic Moisture Sorption Analyzer (ProUmid GmbH & Co. KG, Germany). The sample was placed into a tared sample chamber and automatically weighed.
Sample temperature 25° C.
Cycle: Full cycle
Adsorption 0, 10, 20, 30, 40, 50, 60, 70, 80, 90
Desorption 80, 70, 60, 50, 40, 30, 20, 10, 0

### Example 1E: Proton Nuclear Magnetic Resonance (¹H-NMR)

¹H-NMR was performed using Bruker AVANCE III HD 300 or 400 equipped with automatic sampler (SampleXpress 60), using d⁶-DMSO as solvent.

### Example 1F: High Performance Liquid Chromatography (HPLC)

HPLC analysis was performed with an Agilent HPLC 1260 series instrument.
Column: Ascentis Express C18 4.6*100 mm, 2.7 μm
Mobile Phase A: 0.05% TFA in H₂O B: 0.05% TFA in ACN
Gradient (T/B %): 0/10, 6.0/60, 8.0/90, 10.0/90, 10.1/10, 13.0/10
Column Temperature: 40° C.
Detector (wave: DAD (254 nm)
length)
Flow Rate: 1.8 mL/min
Injection Volume: 5 μL
Run Time: 13 minutes
Post Time: 0 minute
Diluent: ACN:Water=1:1 (V: V)

### Example 2: Characterization of Starting Material

A single batch of Compound 1, as a light-yellow solid, (2.66 g, 99.78% purity), was used as the starting material for the polymorph screen. The material was mostly rod-like crystal with low crystallinity and particle size 10-20 μm. XRPD characterization revealed the material to be a mixture of Form I and Form II which converted to Form I after heating to 170° C.

### Example 3: Polymorph Screen

Various techniques were evaluated for producing crystalline material.

### Example 3A: Evaporative Crystallization

Evaporative crystallization studies were performed in THF, methanol, acetone, isopropanol and dichloromethane (i.e. solvents providing solubility >3 mg/mL), under fast and slow evaporation rates:
Fast: solutions dried by nitrogen purging at RT (˜25° C.)
Slow solutions evaporated to dryness in a fume hood at RT (˜25° C.)

Solubility was measured by HPLC and the residual solid, after evaporation, was analyzed by XRPD. In all cases, only amorphous material was obtained.

### Example 3B: Slurry

Starting material was added to 13 single solvents, and the resulting suspensions stirred for 3 days at RT (˜24° C.) or 50° C. Any solids obtained were characterized and the results are summarized in Table 1 (loading concentrations are in mg/mL). Forms II, III, IV and V were isolated as shown.

TABLE 1
Slurry Study in Single Solvents

| | Room Temp | | 50° C. | |
|---|---|---|---|---|
| | Loading | | Loading | |
| Solvent | conc | Form | conc | Form |
| Methanol | 60 | II | 120 | II |
| Ethanol | 30 | II | 120 | II |
| 2-Propanol | 60 | III | n/a | n/a |
| n-Butanol | 60 | III | n/a | n/a |
| Acetone | 60 | III | n/a | n/a |
| 2-Butanone | 60 | III | n/a | n/a |
| Ethyl acetate | 60 | III | 120 | I + III |
| Isopropyl acetate | 60 | III | 120 | III |
| Heptane | 30 | I | 30 | I |
| t-Butyl methyl ether | 30 | IV | 60 | IV |
| Toluene | 30 | V | 60 | V |
| Acetonitrile | 30 | III | 60 | I + III |
| Water | 30 | Initial Solid | 30 | I + III |

### Example 3C: Cooling Crystallization

Starting material (˜20 mg) was weighed into vials and solvent added resulting in nearly clear, saturated solutions or suspensions which were stirred at 50° C. These were then cooled to room temperature (˜24° C., slow cooling) or the filtrate was placed in a fridge directly (2-8° C., fast cooling). Solids were obtained from methanol and ethanol; cooling of these solids resulted in Form II. All other conditions resulted in solutions. The results are presented in solvents (Table 2).

TABLE 2
Cooling Crystallization

| Solvent | Conc. (mg/mL) | Fast cooling | Slow cooling |
|---|---|---|---|
| Methanol | 60 | Form II | Form II |
| Ethanol | 60 | Similar to Form II | Similar to Form II |
| 2-Propanol | 30 | Solution | Solution |
| n-Butanol | 30 | Solution | Solution |
| Acetone | 60 | Solution | Solution |
| 2-Butanone | 60 | Solution | Solution |
| Ethyl acetate | 30 | Solution | Solution |

### Example 3D: Anti-Solvent Precipitation

Solvent/anti-solvent experiments were performed in 12 systems. Solvents providing high solubility include DMSO, THA, acetone, ethyl acetate, 2-Butanone and IPA. Solvents providing low solubility include IPAc, ACN, MTBE, Heptane were selected as anti-solvents. Starting material (˜20 mg) was dissolved in solvent to prepare a saturated solution. After filtration, anti-solvents were gradually added to the filtrates, in 20-100 L aliquots, until turbidity was observed or 10V was reached at RT (˜25° C.). If precipitation occurred, products were characterized accordingly, and the results shown in Table 3.

Form I was obtained from DMSO/Water (1:1), EtOc/heptane(1/3) and IPA/water (1/3).

Form II was obtained from Acetone/water (1/2).

Form VI was obtained from 2-Butanone/water (1/2). The remaining conditions either remained as solutions or provided insufficient solid for analysis.

TABLE 3
Anti-solvent Precipitation

| Solvent | Anti-solvent | V1/ml | V2/ml | Result |
|---|---|---|---|---|
| DMSO | Water | 0.1 | 0.1 | Form I |
| | IPAC | | 1 | Solution |
| | Acetonitrile | | 1 | Solution |
| THF | MTBE | 1 | 6 | Solution |
| | Water | | 3 | Insufficient solid |
| | Heptane | | 4 | Insufficient solid |
| Acetone | Water | 0.25 | 0.5 | Similar to Form II |
| Ethyl acetate | Heptane | 0.5 | 1.5 | Form I |
| 2-Butanone | Water | 0.25 | 0.5 | Form VI |
| 2-Propanol | Water | 0.5 | 1.5 | Form I |

### Example 4: Characterization of Polymorphic Forms Identified

Seven crystal Forms were identified and assigned as Forms I, II, III, IV, V, VI and VII.

### Form I

Form I, an irregular shaped crystal with high crystallinity and fine particle size, was obtained from a heptane slurry or by heating the starting material to ˜170° C. The XRPD of Form I is shown in FIG. 1 and the significant peaks from the XRPD trace are listed below in Table 4:

TABLE 4
Form I, XRPD Peak List

| Pos. | Height | Rel. Int. |
|---|---|---|
| [°2θ] | [cts] | [%] |
| 4.5826 | 76.34 | 0.79 |
| 8.0416 | 6711.84 | 69.09 |
| 8.7928 | 35.56 | 0.37 |
| 9.2011 | 202.53 | 2.08 |
| 10.4093 | 1511.64 | 15.56 |
| 10.7033 | 1706.53 | 17.57 |
| 12.5767 | 1807.83 | 18.61 |
| 13.3485 | 4051.12 | 41.70 |
| 13.9620 | 984.12 | 10.13 |
| 14.1981 | 1612.85 | 16.60 |
| 15.4985 | 419.08 | 4.31 |
| 16.1506 | 888.28 | 9.14 |
| 16.9181 | 1519.47 | 15.64 |
| 17.9834 | 1622.60 | 16.70 |
| 18.3285 | 1389.37 | 14.30 |
| 18.5159 | 2351.47 | 24.21 |
| 18.8981 | 677.69 | 6.98 |
| 19.3677 | 1120.63 | 11.54 |
| 20.4516 | 9714.32 | 100.00 |
| 20.9129 | 2297.24 | 23.65 |
| 21.4948 | 419.09 | 4.31 |
| 22.2265 | 672.24 | 6.92 |
| 23.0747 | 314.32 | 3.24 |
| 23.4605 | 325.73 | 3.35 |
| 23.9244 | 823.70 | 8.48 |
| 24.3213 | 1733.15 | 17.84 |
| 25.3546 | 365.38 | 3.76 |
| 26.1897 | 1031.78 | 10.62 |
| 26.6395 | 336.28 | 3.46 |
| 27.8792 | 156.81 | 1.61 |
| 28.5335 | 498.67 | 5.13 |
| 29.7030 | 1025.71 | 10.56 |
| 30.4180 | 200.11 | 2.06 |
| 31.3475 | 514.24 | 5.29 |
| 31.6463 | 176.95 | 1.82 |
| 32.2271 | 46.93 | 0.48 |
| 32.7821 | 176.23 | 1.81 |
| 33.2241 | 236.80 | 2.44 |
| 33.6051 | 124.87 | 1.29 |
| 34.4408 | 181.67 | 1.87 |
| 36.1194 | 42.70 | 0.44 |
| 37.1119 | 131.90 | 1.36 |
| 37.8402 | 278.29 | 2.86 |

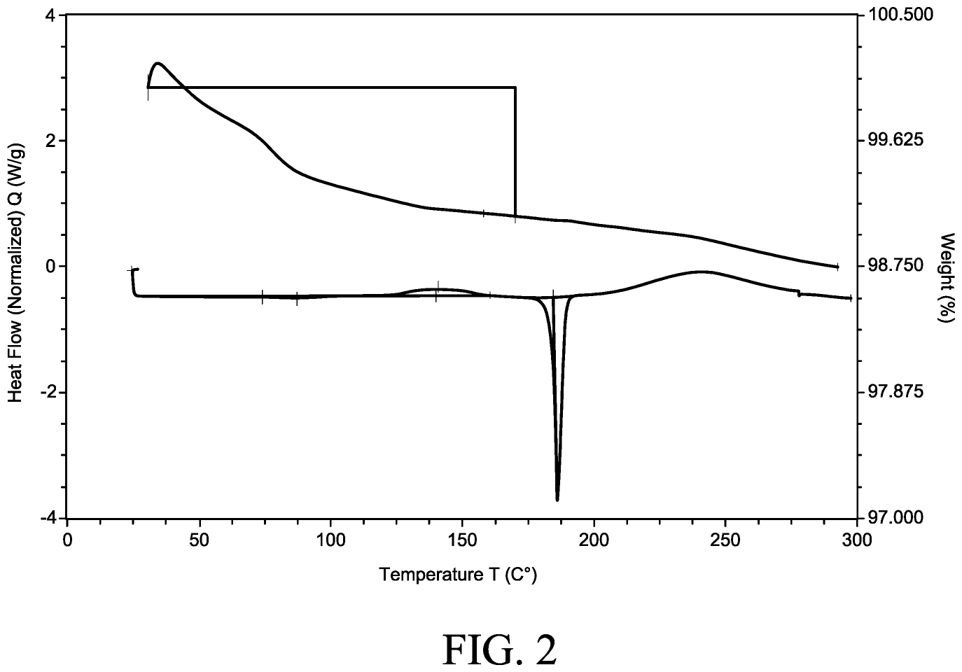

There was a 0.6% weight loss before 165° C. in the TGA profile (FIG. 2).

### Form II

Form II is an irregular crystal with high crystallinity obtained by slurry or cooling from methanol or ethanol. The XRPD of Form II is shown in FIG. 3 and the significant peaks from the XRPD trace are listed below in Table 5:

TABLE 5
Form II, XRPD Peak List

| Pos. | Height | Rel. Int. |
|---|---|---|
| [°2θ] | [cts] | [%] |
| 4.2759 | 230.65 | 2.02 |
| 7.2486 | 2227.47 | 19.48 |
| 7.7928 | 1634.70 | 14.29 |
| 8.5794 | 691.34 | 6.04 |
| 8.9927 | 3711.79 | 32.45 |
| 10.9808 | 86.85 | 0.76 |
| 11.5687 | 868.61 | 7.59 |
| 12.0619 | 6933.85 | 60.62 |
| 12.8265 | 195.46 | 1.71 |
| 13.5692 | 729.25 | 6.38 |
| 14.2821 | 484.29 | 4.23 |
| 14.5609 | 520.52 | 4.55 |
| 15.0510 | 179.57 | 1.57 |
| 15.7491 | 7232.04 | 63.23 |
| 16.1608 | 313.81 | 2.74 |
| 16.4422 | 847.63 | 7.41 |
| 17.1354 | 550.58 | 4.81 |
| 18.1925 | 2587.62 | 22.62 |
| 18.6530 | 162.16 | 1.42 |
| 19.8633 | 527.16 | 4.61 |
| 20.9566 | 11437.45 | 100.00 |
| 22.0615 | 733.73 | 6.42 |
| 23.1175 | 121.16 | 1.06 |
| 23.7645 | 215.07 | 1.88 |
| 24.2411 | 4009.36 | 35.05 |
| 24.7148 | 564.58 | 4.94 |
| 25.4409 | 147.69 | 1.29 |
| 26.2220 | 400.92 | 3.51 |
| 27.7788 | 436.87 | 3.82 |
| 29.1169 | 1637.56 | 14.32 |
| 30.3056 | 306.21 | 2.68 |
| 31.1251 | 86.57 | 0.76 |
| 31.7892 | 192.45 | 1.68 |
| 32.3675 | 1289.21 | 11.27 |
| 33.3397 | 40.95 | 0.36 |
| 34.9932 | 272.14 | 2.38 |
| 35.8143 | 228.91 | 2.00 |
| 36.8235 | 92.48 | 0.81 |
| 38.2706 | 57.33 | 0.50 |
| 39.6679 | 101.02 | 0.88 |

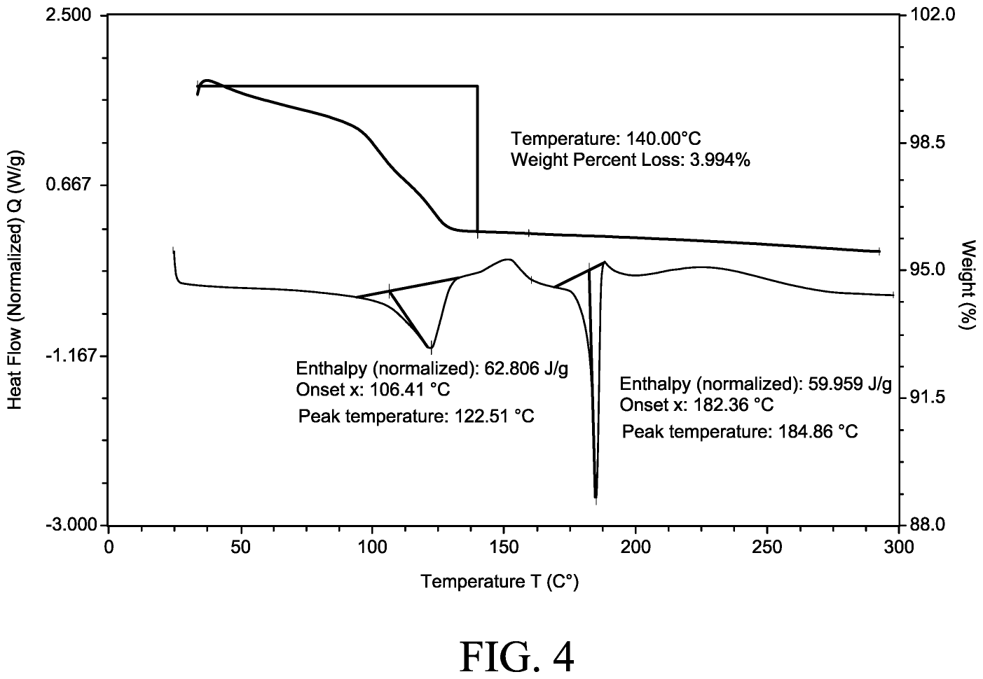

Multiple thermal events were observed in the DSC curve (FIG. 4). The first endothermic peak was the dehydration/de-solvation of Form II and the second endothermic peak was the melting of Form I. Approx. 4.0% weight loss prior to 140° C. was observed in the TGA profile, and 1.7% ethanol was detected by NMR.

### Form III

Form III is an irregular crystal with high crystallinity obtained from slurry in single solvent. The XRPD of Form III is shown in FIG. 5 and the significant peaks from the XRPD trace are listed below in Table 6:

TABLE 6
Form III, XRPD Peak List

| Pos. | Height | Rel. Int. |
|---|---|---|
| [°2θ] | [cts] | [%] |
| 8.6554 | 2139.84 | 52.72 |
| 9.0719 | 1055.79 | 26.01 |
| 9.5455 | 997.89 | 24.58 |
| 10.2543 | 196.06 | 4.83 |
| 12.0967 | 1162.92 | 28.65 |
| 12.4933 | 1381.60 | 34.04 |
| 13.0330 | 2462.09 | 60.66 |
| 13.5006 | 671.25 | 16.54 |
| 13.9691 | 604.14 | 14.88 |
| 15.1465 | 1309.06 | 32.25 |
| 16.5026 | 1989.37 | 49.01 |
| 16.8473 | 109.68 | 2.70 |
| 17.9249 | 143.36 | 3.53 |
| 18.7262 | 663.06 | 16.34 |
| 19.0534 | 1034.59 | 25.49 |
| 19.2206 | 2428.75 | 59.83 |
| 19.6384 | 803.54 | 19.80 |
| 20.3115 | 166.47 | 4.10 |
| 20.7586 | 2004.44 | 49.38 |
| 21.2802 | 281.65 | 6.94 |
| 21.5348 | 300.49 | 7.40 |
| 22.1630 | 4059.10 | 100.00 |
| 22.9115 | 1554.30 | 38.29 |
| 23.7892 | 161.60 | 3.98 |
| 24.4164 | 716.30 | 17.65 |
| 24.7188 | 98.53 | 2.43 |
| 25.2054 | 74.11 | 1.83 |
| 25.5478 | 102.08 | 2.51 |
| 26.3057 | 173.14 | 4.27 |
| 26.6660 | 784.04 | 19.32 |
| 27.2403 | 148.36 | 3.66 |
| 27.8135 | 363.76 | 8.96 |
| 28.5607 | 108.87 | 2.68 |
| 29.0224 | 536.13 | 13.21 |
| 30.0978 | 51.56 | 1.27 |
| 31.4197 | 227.20 | 5.60 |
| 32.2077 | 116.41 | 2.87 |
| 32.9694 | 103.99 | 2.56 |
| 33.4510 | 146.62 | 3.61 |
| 33.8089 | 135.27 | 3.33 |
| 34.1868 | 100.72 | 2.48 |
| 35.7088 | 136.64 | 3.37 |
| 37.1176 | 47.83 | 1.18 |
| 38.4358 | 134.08 | 3.30 |

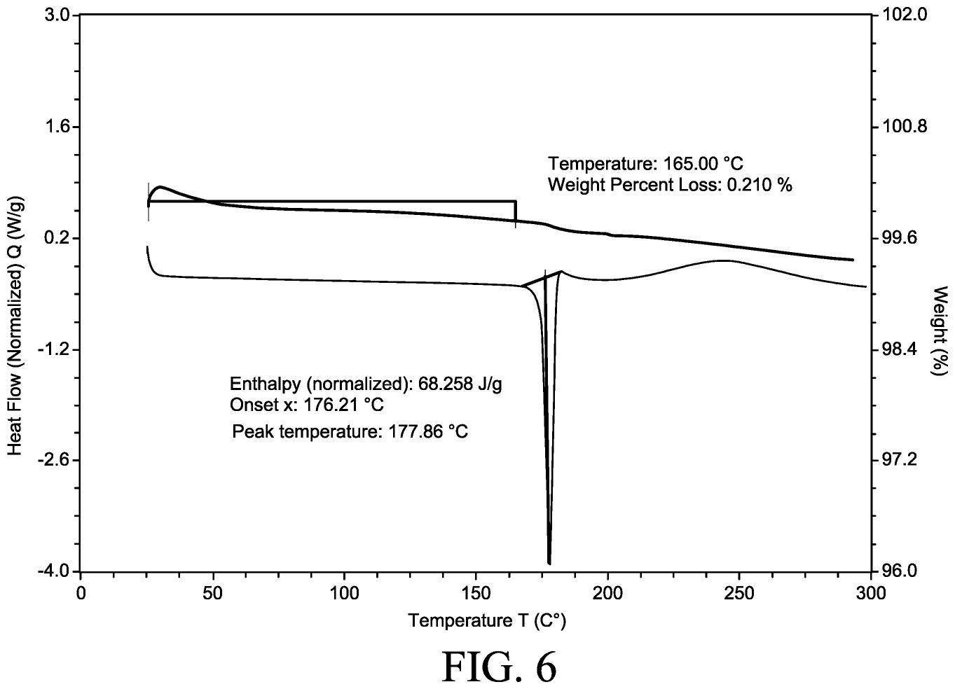

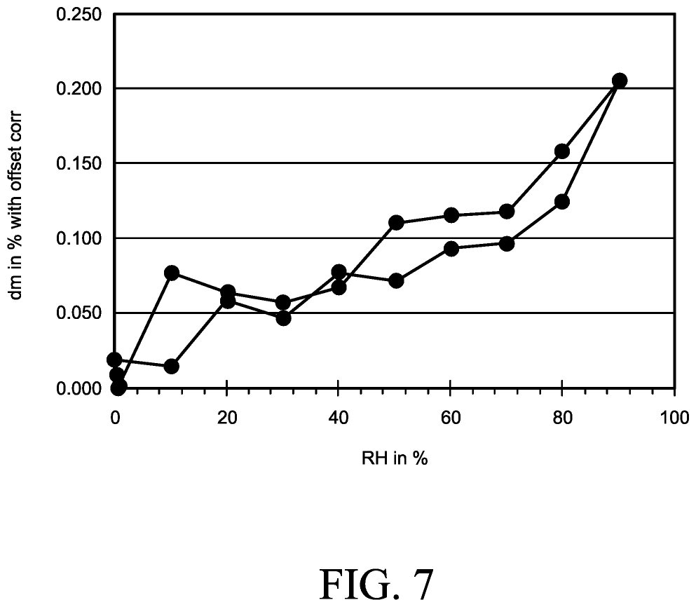

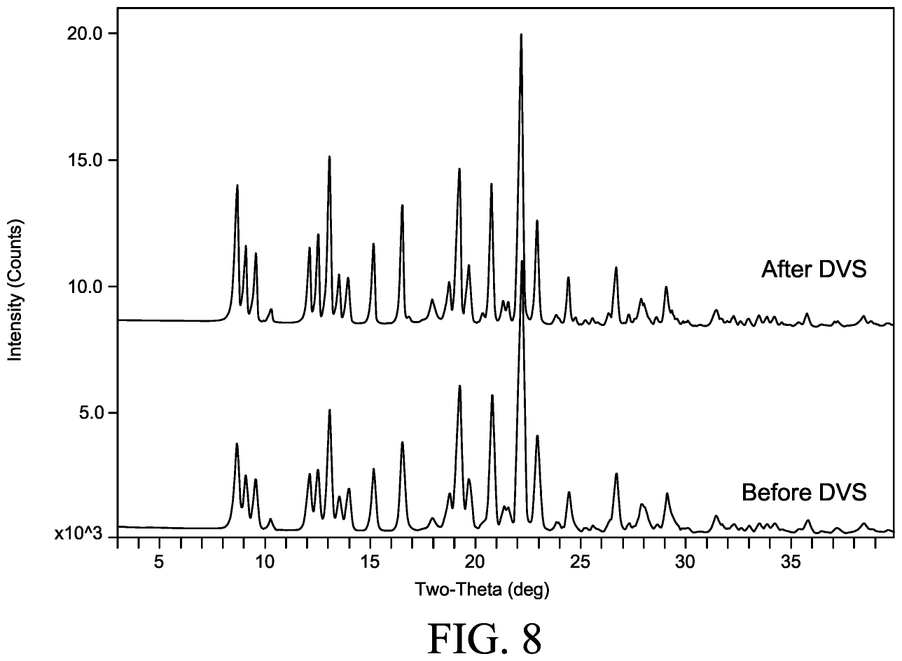

One melting peak was observed in the DSC curve and there was no obvious weight loss between RT to 165° C. in the TGA profile (FIG. 6), indicating that Form III was an anhydrate form. The result of competition slurry of Form I and Form III indicates that Form III is the most stable Form from RT to 80° C. DVS data showed Form III was non-hygroscopic (<0.5% weight gain up to 90% RH, see FIG. 7) and the crystal Form remained unchanged after DVS testing (FIG. 8).

### Form IV

Form IV is an irregular crystal with high crystallinity, only obtained from an MTBE slurry. The XRPD of Form IV is shown in FIG. 9 and the significant peaks from the XRPD trace are listed below in Table 7:

TABLE 7
Form IV, XRPD Peak List

| Pos. | Height | Rel. Int. |
|---|---|---|
| [°2θ] | [cts] | [%] |
| 8.3694 | 8593.54 | 60.86 |
| 8.6027 | 777.17 | 5.50 |
| 9.3238 | 3513.62 | 24.88 |
| 9.4292 | 4084.87 | 28.93 |
| 10.1529 | 103.27 | 0.73 |
| 11.9598 | 1160.57 | 8.22 |
| 12.6229 | 156.44 | 1.11 |
| 13.3599 | 490.78 | 3.48 |
| 13.9360 | 1886.75 | 13.36 |
| 14.4331 | 241.16 | 1.71 |
| 15.3077 | 1002.00 | 7.10 |
| 16.2354 | 423.50 | 3.00 |
| 16.8979 | 1012.84 | 7.17 |
| 17.3121 | 1314.17 | 9.31 |
| 17.7114 | 632.48 | 4.48 |
| 18.1902 | 1906.80 | 13.50 |
| 18.8538 | 5503.03 | 38.97 |
| 19.1307 | 899.95 | 6.37 |
| 19.7438 | 14121.21 | 100.00 |
| 20.2418 | 3670.82 | 26.00 |
| 21.3210 | 455.48 | 3.23 |
| 21.5845 | 1863.95 | 13.20 |
| 22.0156 | 806.90 | 5.71 |
| 22.4693 | 557.19 | 3.95 |
| 23.0501 | 99.33 | 0.70 |
| 23.6897 | 1220.19 | 8.64 |
| 24.0848 | 437.34 | 3.10 |
| 24.6494 | 109.14 | 0.77 |
| 25.4090 | 3165.36 | 22.42 |
| 25.7634 | 635.95 | 4.50 |
| 26.8514 | 1291.53 | 9.15 |
| 27.3691 | 665.46 | 4.71 |
| 27.7683 | 601.26 | 4.26 |
| 28.0521 | 542.82 | 3.84 |
| 28.4714 | 251.18 | 1.78 |
| 28.7353 | 206.98 | 1.47 |
| 29.7532 | 34.67 | 0.25 |
| 30.7052 | 276.59 | 1.96 |
| 30.9937 | 268.48 | 1.90 |
| 31.4822 | 203.98 | 1.44 |
| 32.1236 | 140.88 | 1.00 |
| 32.3974 | 410.08 | 2.90 |
| 32.7556 | 189.94 | 1.35 |
| 33.4761 | 140.59 | 1.00 |
| 34.1252 | 57.72 | 0.41 |
| 35.0013 | 152.78 | 1.08 |
| 36.1124 | 217.96 | 1.54 |
| 36.4601 | 166.04 | 1.18 |
| 36.9164 | 81.15 | 0.57 |
| 38.2125 | 132.52 | 0.94 |
| 38.7626 | 98.55 | 0.70 |

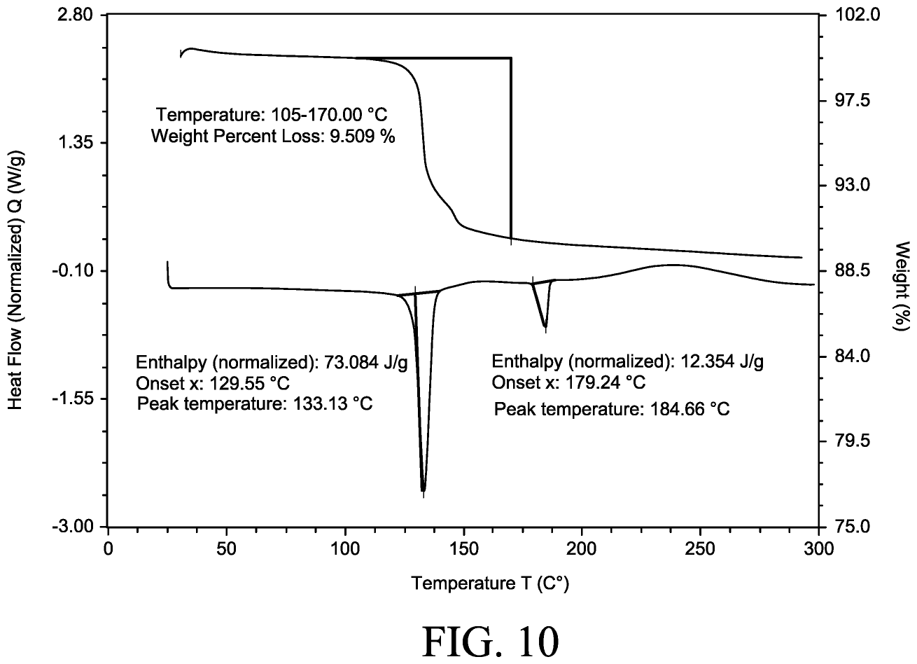

DSC of Form IV (FIG. 10) shows two endothermic peaks before 200° C., corresponding to the de-solvation of Form IV and melting of Form I, respectively. Approx. 9.5% weight loss between 105-170° C. was observed in the TGA profile. 9.8% MTBE was detected by NMR.

### Form V

Form V is an irregular crystal with high crystallinity, only obtainable from a toluene slurry. The XRPD of Form V is shown in FIG. 11 and the significant peaks from the XRPD trace are listed below in Table 8:

TABLE 8
Form V, XRPD Peak List

| Pos. | Height | Rel. Int. |
|---|---|---|
| [°2θ] | [cts] | [%] |
| 6.0772 | 5358.89 | 80.45 |
| 6.4014 | 168.61 | 2.53 |
| 8.4579 | 6660.74 | 100.00 |
| 8.7970 | 623.69 | 9.36 |
| 9.1908 | 840.73 | 12.62 |
| 9.4200 | 1883.66 | 28.28 |
| 9.5857 | 699.76 | 10.51 |
| 9.7579 | 734.51 | 11.03 |
| 10.1868 | 840.05 | 12.61 |
| 10.3836 | 1275.06 | 19.14 |
| 12.1161 | 624.85 | 9.38 |
| 12.8125 | 2027.78 | 30.44 |
| 14.0561 | 142.42 | 2.14 |
| 14.8143 | 835.17 | 12.54 |
| 15.2412 | 449.01 | 6.74 |
| 15.9154 | 481.05 | 7.22 |
| 17.5539 | 1098.74 | 16.50 |
| 17.8750 | 516.45 | 7.75 |
| 18.2766 | 751.18 | 11.28 |
| 18.9810 | 2854.59 | 42.86 |
| 19.1860 | 1159.75 | 17.41 |
| 19.4547 | 2712.97 | 40.73 |
| 20.9150 | 3099.49 | 46.53 |
| 21.1673 | 1273.79 | 19.12 |
| 21.5891 | 1536.98 | 23.08 |
| 22.9266 | 1156.75 | 17.37 |
| 23.2600 | 752.09 | 11.29 |
| 23.9883 | 1380.72 | 20.73 |
| 24.3999 | 969.90 | 14.56 |
| 24.9707 | 527.20 | 7.91 |
| 25.5807 | 1305.33 | 19.60 |
| 26.0308 | 174.70 | 2.62 |
| 26.8697 | 337.42 | 5.07 |
| 27.2564 | 481.83 | 7.23 |
| 27.9364 | 420.36 | 6.31 |
| 28.1873 | 361.18 | 5.42 |
| 28.8615 | 287.27 | 4.31 |
| 29.9363 | 58.08 | 0.87 |
| 30.7528 | 259.53 | 3.90 |
| 31.4762 | 98.97 | 1.49 |
| 32.0388 | 103.60 | 1.56 |
| 32.6665 | 104.08 | 1.56 |
| 33.6635 | 31.69 | 0.48 |
| 34.8100 | 73.74 | 1.11 |
| 35.5896 | 103.40 | 1.55 |
| 36.0074 | 86.14 | 1.29 |
| 36.5456 | 74.84 | 1.12 |
| 37.9884 | 140.18 | 2.10 |
| 38.8789 | 160.73 | 2.41 |
| 39.7259 | 92.25 | 1.38 |

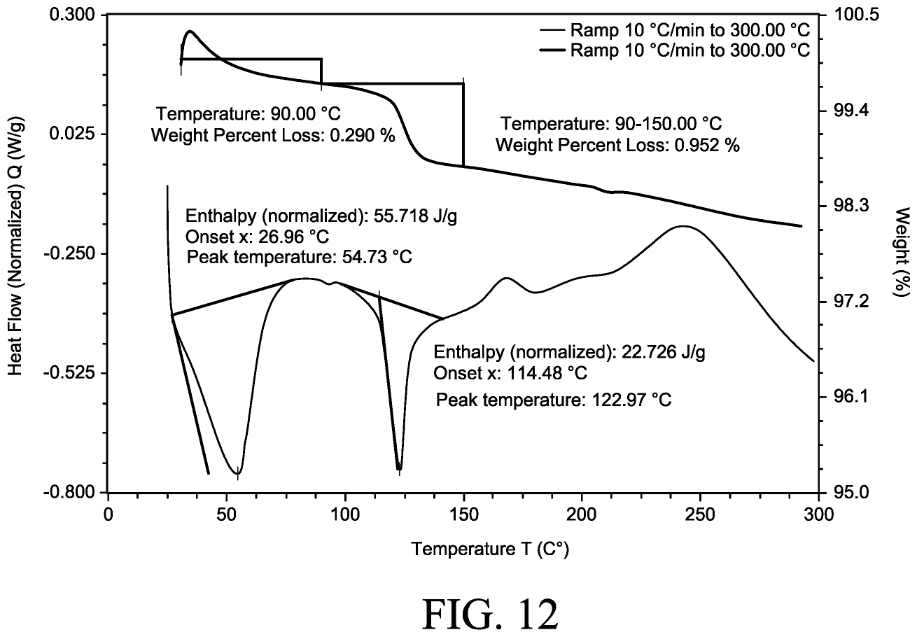

Form V was heated at 85° C. and 155° C., and the resulting solid forms analyzed by PXRD (FIG. 11, upper traces). Form V heated to 85° C., remains unchanged; heating to 155° C., results in formation of the amorphous form. DSC showed two endothermic peaks before 150° C. (FIG. 12). The TGA profile showed approx. 1.0% weight loss between 90-170° C. 1.0% toluene was detected by NMR.

### Form VI

Form VI is an irregular crystal with high crystallinity, obtained from MEK/H₂O anti-solvent crystallization. The XRPD of Form VI is shown in FIG. 13 and the significant peaks from the XRPD trace are listed below in Table 9:

TABLE 9
Form VI, XRPD Peak List

| Pos. | Height | Rel. Int. |
|---|---|---|
| [°2θ] | [cts] | [%] |
| 6.8339 | 87.80 | 0.80 |
| 8.5677 | 1577.23 | 14.45 |
| 10.1404 | 103.69 | 0.95 |
| 11.6455 | 974.55 | 8.93 |
| 11.9246 | 848.25 | 7.77 |
| 13.0868 | 6673.19 | 61.15 |
| 13.7874 | 2725.22 | 24.97 |
| 14.7047 | 24.58 | 0.23 |
| 15.6784 | 1525.82 | 13.98 |
| 16.1217 | 1680.22 | 15.40 |
| 16.8541 | 164.32 | 1.51 |
| 17.5940 | 1826.61 | 16.74 |
| 18.0906 | 642.55 | 5.89 |
| 18.3645 | 2969.74 | 27.21 |
| 20.2179 | 407.65 | 3.74 |
| 20.6765 | 2059.25 | 18.87 |
| 21.7127 | 99.69 | 0.91 |
| 22.0093 | 518.86 | 4.75 |
| 23.7506 | 883.47 | 8.10 |
| 23.9827 | 10912.95 | 100.00 |
| 24.3723 | 838.38 | 7.68 |
| 24.6130 | 721.42 | 6.61 |
| 25.5122 | 2860.73 | 26.21 |
| 26.0909 | 640.10 | 5.87 |
| 26.3973 | 343.77 | 3.15 |
| 26.7363 | 3351.96 | 30.72 |
| 27.0926 | 252.59 | 2.31 |
| 27.8094 | 1105.38 | 10.13 |
| 28.6564 | 97.78 | 0.90 |
| 29.6871 | 563.06 | 5.16 |
| 30.2185 | 289.21 | 2.65 |
| 31.6720 | 53.56 | 0.49 |
| 32.9054 | 139.68 | 1.28 |
| 33.1565 | 182.19 | 1.67 |
| 34.0541 | 105.29 | 0.96 |
| 34.6812 | 76.17 | 0.70 |
| 35.2646 | 124.64 | 1.14 |
| 36.3006 | 132.86 | 1.22 |
| 37.0131 | 319.95 | 2.93 |
| 37.8686 | 87.95 | 0.81 |
| 38.6760 | 72.88 | 0.67 |
| 39.0842 | 75.04 | 0.69 |
| 39.6712 | 178.98 | 1.64 |

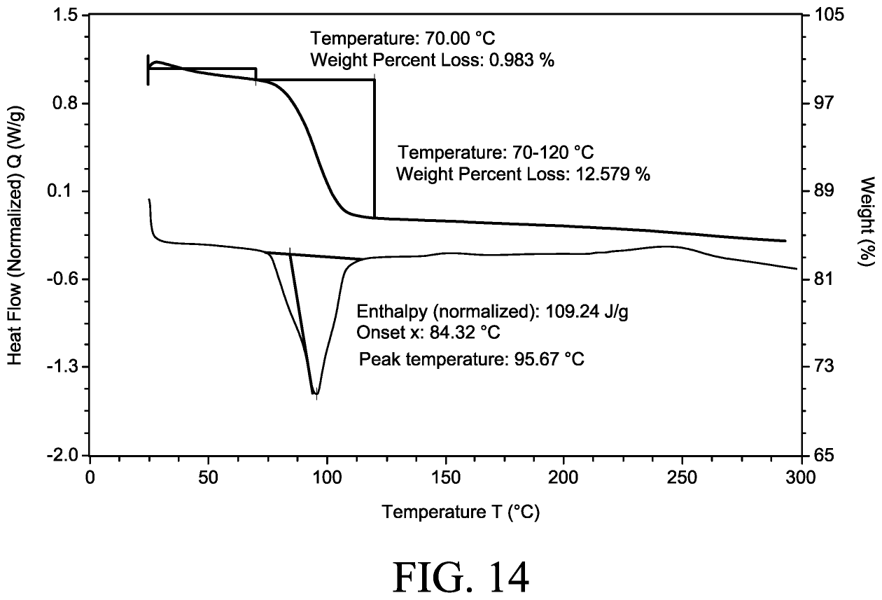

DSC showed abroad endothermic peak (FIG. 14) with 13% weight loss before 120° C. 12% MEK was detected by NMR.

### Form VII

Form VII was observed during isolation of Compound 1 via a heptane/2-methyltetrahydrofuran (2-MeTHF) recrystallization, and determined to be a 2-MeTHF solvate. More specifically, a solution of Compound 1 was dissolved in 2-MeTHF (3 volumes) at 60° C. Heptane antisolvent (4.1 volumes) was then added over 1 h, maintaining the temperature at 60° C. The mixture was then cooled to 20° C. and agitated for 4 hours at 20° C. The resulting solids were isolated by filtration and the wet cake washed with heptane (1.4 volumes), dried for 30 minutes and then dried in a vacuum oven at 55° C. for at least 12 hrs.

The XRPD of Form VII is shown in FIG. 15 and the significant peaks from the XRPD trace are listed below in Table 10:

TABLE 10
Form VII, XRPD Peak List

| Pos. | Height | Rel. Int. |
|---|---|---|
| [°2θ] | [cts] | [%] |
| 6.727 | 73.68 | 1.12 |
| 8.4799 | 6572.16 | 100 |
| 9.4854 | 2759.3 | 41.98 |
| 9.9197 | 86.07 | 1.31 |
| 11.4272 | 48.78 | 0.74 |
| 12.0161 | 895.01 | 13.62 |
| 12.5311 | 180.68 | 2.75 |
| 13.2495 | 318.79 | 4.85 |
| 14.2831 | 507.75 | 7.73 |
| 15.1395 | 133.43 | 2.03 |
| 15.3361 | 281.89 | 4.29 |
| 17.1901 | 762.4 | 11.6 |
| 17.5523 | 648.86 | 9.87 |
| 17.9433 | 268.94 | 4.09 |
| 18.3373 | 573.07 | 8.72 |
| 18.8407 | 1196.13 | 18.2 |
| 19.0691 | 2673.33 | 40.68 |
| 19.7285 | 4160.72 | 63.31 |
| 20.2268 | 3136.55 | 47.72 |
| 21.2664 | 250.46 | 3.81 |
| 21.5376 | 721.46 | 10.98 |
| 22.4347 | 93.03 | 1.42 |
| 23.486 | 364 | 5.54 |
| 24.2132 | 152.83 | 2.33 |
| 24.6106 | 217.41 | 3.31 |
| 25.2572 | 1279.1 | 19.46 |
| 25.6894 | 295.39 | 4.49 |
| 26.395 | 198.79 | 3.02 |
| 26.7047 | 940.16 | 14.31 |
| 27.1163 | 92.52 | 1.41 |
| 27.4747 | 232.91 | 3.54 |
| 27.8122 | 166.13 | 2.53 |
| 28.1719 | 200 | 3.04 |
| 28.8013 | 170.49 | 2.59 |
| 29.3516 | 98.54 | 1.5 |
| 30.7728 | 70.83 | 1.08 |
| 31.6849 | 127.02 | 1.93 |
| 32.2782 | 281.31 | 4.28 |
| 33.4385 | 31.82 | 0.48 |
| 34.4842 | 35.28 | 0.54 |
| 34.8513 | 72.24 | 1.1 |
| 35.5474 | 34.22 | 0.52 |
| 36.7698 | 48.86 | 0.74 |
| 37.4812 | 90.63 | 1.38 |
| 39.3539 | 55.26 | 0.84 |

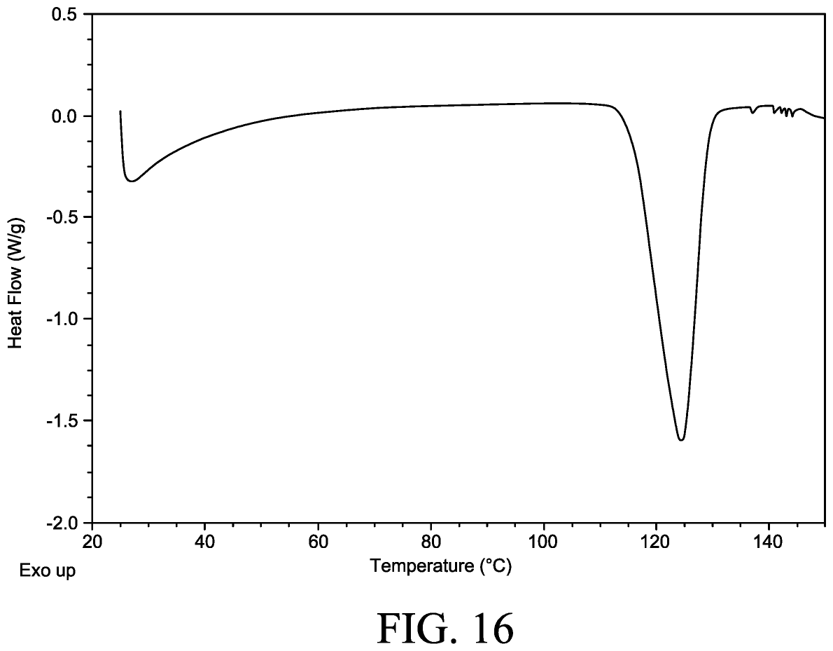

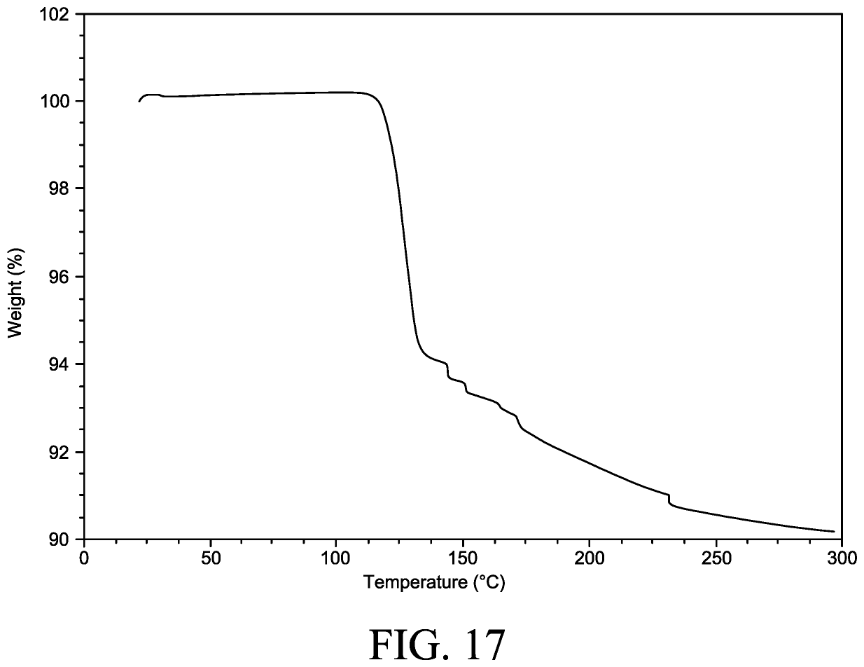

DSC and TGA of Form VII (FIGS. 16 and 17, respectively) shows one endothermic at 122-124° C., corresponding to the de-solvation of Form VII. Approximately 6.6% weight loss between 102-105° C. was observed in the TGA profile. 9.9% 2-methyl-THG was detect by NMR.

### Summary of Solid Forms Identified

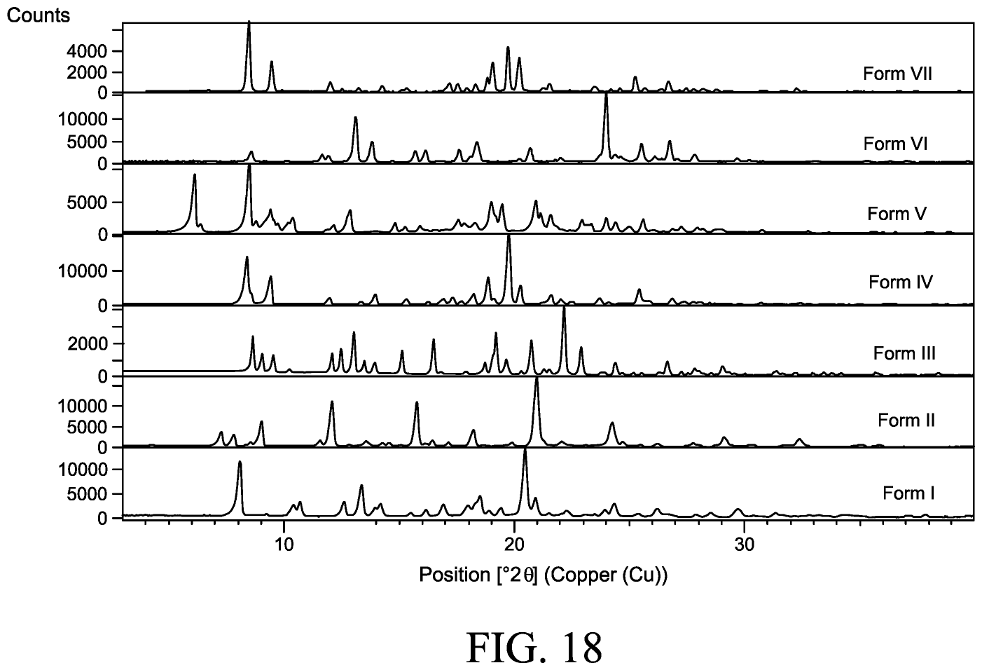

Seven new crystal Forms were identified and assigned as Form I-VII. FIG. 18 shows the XRPD overlay of all seven forms. Forms I and III are anhydrates; Forms II, IV, V, VI, and VII are solvates or hydrates. Form II and Form IV convert to Form I upon heating, see Table 11. Table 12 shows characterization data for the various forms.

TABLE 11
Formation and conversion of Forms I-VIIVI

| Form | Comment |
|---|---|
| S material | Mixture of forms, converted to Form I at 170° C. |
| I | Obtained from slurry in heptane, anti-solvent and heating |
| II | Converted to Form I after dehydration |
| III | Obtained by slurry in single solvents |
| V | Converted to Form I after de-solvation |
| V | Obtained from toluene slurry |
| VI | Obtained from anti-solvent crystallization (MEK/water) |
| VII | Obtained from anti-solvent crystallization (2-MeTHF/heptane) |

TABLE 12
Characterization of Forms I-VII

| | | | DSC | TGA |
|---|---|---|---|---|
| Form | | Crystal Form | Endo Onset/Peak, ΔH | Wt. loss/@T |
| SM | | Rod + irregular | 74/87° C., 4 J/g and | 0.9%/25-170° C. |
| | | | 140/141° C., 14 (Exo); | |
| | | | 185/186° C., 67 J/g | |
| I | Anhydrate | Irregular | 183/184° C., 54 J/g | 0.6%/25-165° C. |
| II | Hydrate | Irregular | 106/123° C., 63 J/g and | 4.0%/25-140° C. |
| | | | 182/185° C., 60 J/g | |
| III | Anhydrate | Irregular | 176/178° C., 68 J/g | 0.2%/25-165° C. |
| IV | MTBE Solvate | Irregular | 130/133° C., 73 J/g and | 9.5%/105-170° C. |
| | | | 179/185° C., 12 J/g | |
| V | Toluene | Irregular | 27/55° C., 56 J/g and | 0.3%/25-90° C. and |
| | Solvate | | 114/123° C., 23 J/g | 1.0%/90-150° C. |
| VI | MEK Solvate | Irregular | 78/94° C., 122 J/g | 13%/65-120° C. |
| VII | 2-MeTHF | Plate | 112-115° C./120-124° C., | ~6-10%/~102-150° C. |
| | Solvate | | 78-81 J/g | |

### Inter-Conversion Study

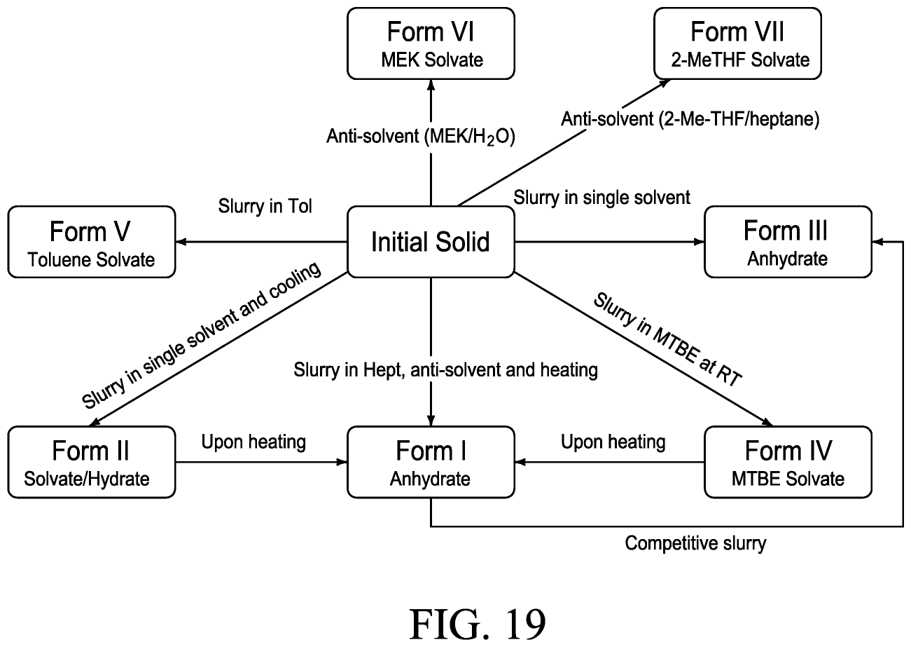

Equal amounts of Form I and Form III were mixed in water and heptane to form slurries and stirred at RT (˜25° C.) or 80° C. Residual solids were isolated and characterized and showed conversion to all Form III. Thus, Form III was considered more stable than Form I. A proposed conversion map of the seven crystal Forms is shown in FIG. 19.

### Example 5: Preparation & Characterization of Form III

### Example 5A: 120 mg PREPARATION of Form III

A suspension of starting material (150 mg) in isopropyl acetate (2.5 mL) was stirred at room temperature for 3 days. The resultant solid was isolated by filtration to provide irregular shaped crystals (120 mg; 80% yield). XRPD analysis of the resultant material, as compared to reference Form III material confirms the material was Form III. This Form III material was used for the studies described below.

### Example 5B: DSC & TGA Analysis

Thermal treatment of Compound 1, Form III was conducted using DSC as follows:
Equilibrate at 25° C.;
Ramp 10° C./min to 190° C.;
Equilibrate at −40° C.; and
Ramp 5° C./min to 300° C.

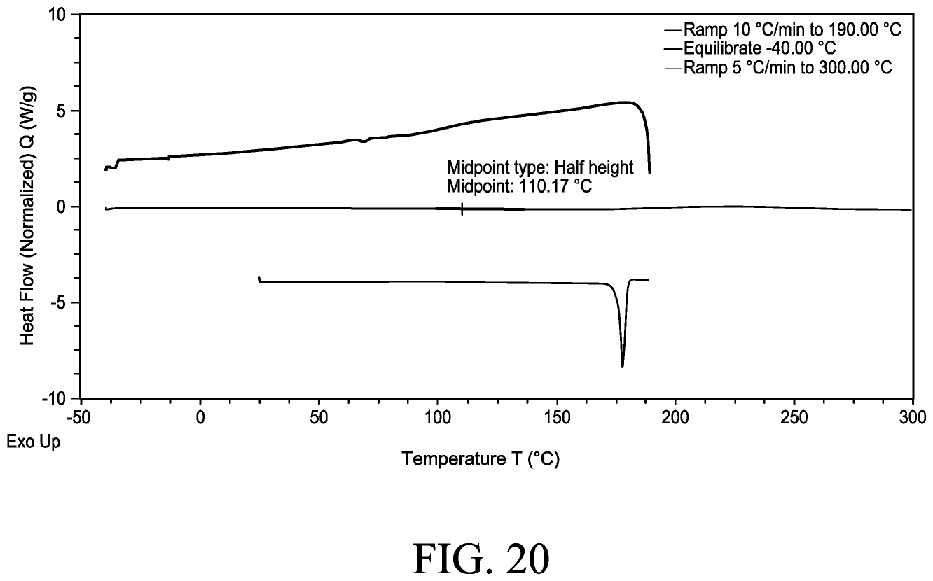

FIG. 20 shows the resultant DSC curve, wherein the bottom curve is ramp 10° C./min to 190° C.; middle curve is equilibration at −40° C. and the top curve is ramp 5° C./min to 300° C. The glass transition temperature is 110° C. An endothermic peak with onset temperature of 175.8° C. was detected.

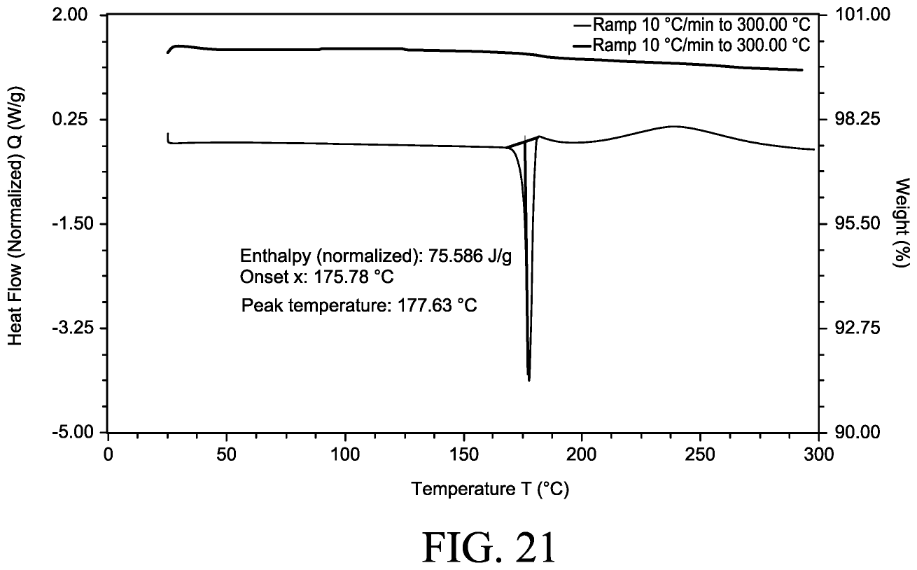

FIG. 21 shows an overlay of TGA and DSC Thermograms of Form III. No obvious weight loss before melting was observed in the TGA profile.

### Example 5C: Solid-State Stability

The solid-state stability of Compound 1, Form III was examined in duplicate (sample 1 and sample 2) for 7 days under the conditions below and the resultant solids analyzed by XRPD:
40° C./75% relative humidity (open); and
60° C. (capped).

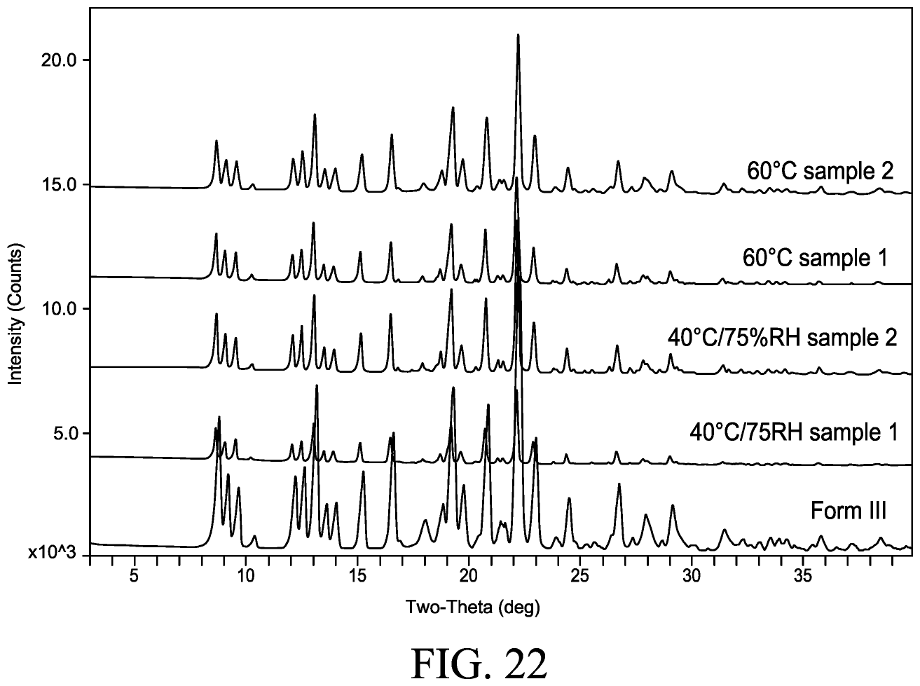

FIG. 22 shows the XRPD patterns of the starting material and the material isolated after exposure to the above conditions and shows that Form III was both chemically and physically stable under the conditions assessed.

The solid-state stability of Compound 1, Form III was also examined for 3 months under the conditions below:
40° C./75% relative humidity; and
25° C./60% relative humidity

Form III was stable under the conditions assessed.

### Example 5D: Solubility Testing

The approximate solubility of the initial solid was determined using a solvent addition method via visual assessment of samples. The results are summarized in Table 13. The starting material is freely soluble in DMSO (>250 mg/mL), sparingly soluble in THE and dioxane (>20 mg/mL) and, has very low solubility in MTBE, water, and n-heptane (<0.6 mg/mL).

TABLE 13
Preliminary Solubility Data

| Solvent | Solubility(mg/mL) |
|---|---|
| Dimethyl sulfoxide (DMSO) | >250 |
| Tetrahydrofuran (THF) | ~25 |
| 1,4-dioxane | ~20 |
| 2-Butanone (MEK) | ~8.3 |
| Acetone | ~7.1 |
| Methanol | ~7.1 |
| Ethanol | ~5 |
| 2-Propanol (IPA) | ~4.2 |
| n-Butanol (NBA) | ~2.3 |
| Ethyl acetate | ~2.0 |
| Acetonitrile | ~1.7 |
| Toluene | ~1.3 |
| Isopropyl acetate | ~1.5 |
| tert-Butyl methyl ether (MTBE) | ~0.6 |
| n-Heptane | <0.6 |
| Water | <0.6 |

Values are rounded to nearest whole number and reported as “<” if dissolution was not observed, and as “>” if dissolution occurred after addition of first aliquot.

The Form III was non-hygroscopic and chemically and physically stable at 40° C./75% humidity and 60° C. for 1 week.

Form III remained unchanged after grinding while the crystallinity decreased slightly.

Form III can be prepared by slurry from single solvents, such as IPAC, IPA and ACN.

### Characterization of Compound 1, Form III

In each of the following studies (Examples 6-10), the starting material was Compound 1 (free form, not salt) Form III. The material was a light yellow solid, comprising non-hygroscopic irregular shaped crystal with aggregation. The same analytical methods as described in Example 1 were employed.

### Example 6: pKa Determination

pKₐ was assessed using Sirius T3 titrator. The acidic and basic sites were very weak, and undetectable by Sirius T3. The mean pKa individual results (for up titrations performed at 25° C.) were as follows:

| TitrationIonic Strength | Chi squared |
|---|---|
| Points 3-510.168 | 0.6254 |
| Points 52-102 | 0.183 0.8024 |
| Points 103-153 | 0.196 0.6051 |

### Example 7: Solubility Testing in Vehicle

Vehicle solutions were prepared as follows:

| Vehicle | Procedure |
|---|---|
| 10% HP-β-CD | 1.0 g HP-β-CD was dissolved in water and diluted |
| | to 10 mL |
| 1% Tween 80: | 0.1 g of Tween 80 was dissolved in water and diluted |
| | to 10 mL |
| 5% TPGS: | 0.5 g of TPGS was dissolved in water and diluted |
| | to 10 mL |
| 1% polox188: | 0.1 g of polox188 was dissolved in water and diluted |
| | to 10 mL |
| 10% SBE-β-CD: | 1 g of SBE-β-CD was dissolved in water and diluted |
| | to 10 mL |
| 1% polox407: | 0.1 g of polox407 was dissolved in water and diluted |
| | to 10 mL |
| 5% PVP VA64: | 0.5 g of PVP VA64 was dissolved in water and diluted |
| | to 10 mL |
| 5% PVP K30: | 0.5 g of PVP K30 was dissolved in water and diluted |
| | to 10 mL |
| 1% SLS: | 0.1 g of 1% SLS was dissolved in water and diluted |
| | to 10 mL |
| 20% TPGS | 1.0 g of TPGS was dissolved in water and diluted |
| | to 5 mL |
| PEG400/TPGS | 10 g of TPGS was dissolved in 30 g PEG400 at 45° C. |
| (W/W = 3/1): | |
| 20% SBE-β-CD: | 1 g of SBE-β-CD was dissolved in water and diluted |
| | to 5 mL |

### Example 8A: Solubility Testing

Solubility was determined at room temperature for 24 and 72 hours and at 45° C. for 4 and 24 hours. About 30/100 mg Compound 1 was added to vehicle (2 mL) and the mixture stirred at room temperature for 24 or 72 hours. The suspensions were filtered or centrifuged, and the filtrate analyzed by HPLC. Solubility and pH results are presented in Tables 14 and 15. After 24 hours, high solubility (>50 mg/mL) was observed in Cremophor HS 15, Gelucire 44/14, Gelucire 48/16 and PEG400/TPGS (3/1). After 72 hours, high solubility (>50 mg/mL) was observed in PG, PEG400, Capryol 90, and Labrasol.

TABLE 14
Solubility at Room Temperature

| | Loading Conc | pH | | Solubility (mg/mL) | |
|---|---|---|---|---|---|
| Vehicle | (mg/mL) | 24 h | 72 h | 24 h | 72 h |
| 10% HP-B-CD | 15 | 6.7 | 6.7 | 0.23 | 0.22 |
| 10% SBE-B-CD | 15 | 6.5 | 6.5 | 1.60 | 1.61 |
| Sesame oil | 15 | 6.5 | 6.5 | 0.73 | 0.76 |
| Miglyol 810 | 15 | 5.8 | 5.8 | 2.16 | 2.67 |
| Phosal 50PG | 50 | 5.8 | 5.8 | 15.65 | 23.78 |
| 1% Tween 80 | 15 | 6.3 | 6.3 | 0.16 | 0.17 |
| 10% TPGS | 15 | 5.3 | 5.3 | 2.03 | 2.04 |
| 1% polox188 | 15 | 5.7 | 5.7 | 0.0002 | 0.0004 |
| 1% polox407 | 15 | 6.7 | 6.9 | 0.004 | 0.001 |
| 5% PVP VA64 | 15 | 4.6 | 4.6 | 0.036 | 0.042 |
| 5% PVP K30 | 15 | 4.0 | 4.0 | 0.008 | 0.060 |
| 1% SLS | 15 | 7.1 | 7.2 | 1.20 | 1.15 |
| Olive oil | 15 | 5.2 | 5.8 | 0.44 | 0.91 |
| PG | 50 | 5.1 | 5.2 | 48.2 | >50 |
| PEG400 | 50 | 5.6 | 6.7 | >50 | >50 |
| EtOH | 50 | 5.9 | 6.2 | 19.4 | 27.6 |
| Cremophor EL | 50 | 8.5 | 8.3 | 19.5 | 33 |
| Capryol 90 | 50 | 6.8 | 6.5 | 47.9 | >50 |
| Labrasol | 50 | 6.9 | 6.4 | >50 | >50 |

TABLE 15
Solubility at 45° C.

| | Loading Conc | pH | | Solubility (mg/mL) | |
|---|---|---|---|---|---|
| Vehicle | (mg/mL) | 4 h | 24 h | 4 h | 24 h |
| Cremophor HS 15 | 50 | 7.6 | 7.5 | >50 | >50 |
| Gelucire 44/14 | 50 | 5.7 | 5.8 | 48.1 | >50 |
| Gelucire 48/16 | 50 | N/A | | 44.3 | >50 |
| PEG400/TPGS(3/1) | 50 | 6.6 | 6.1 | >50 | >50 |
| Oleic acid | 15 | 3.0 | 3.3 | 8.5 | 14.0 |
| Cremophor RH40 | 50 | N/A | | 18.6 | 33 |
| phsal 53 MCT | 15 | 5.3 | 5.1 | 6.4 | >15 |

### Example 8B: Further Solubility Testing

Compound 1 (˜0.5 g) was added to each excipient until visually saturated. The mixtures were incubated on a temperature-controlled mixer with glass beads to facilitating mixing, for at least 48 hours at room temperature (liquid excipients) or 40° C. (semisolid excipients). The samples were then centrifuged using 0.45 μm PVDF filter to separate the liquid and solid portions. The amount of dissolved compound in the filtrate was quantified by HPLC. XRPD analysis of any solid powder confirmed there was no change in form. The solubility results are presented in Table 16 and show Compound 1 had high solubility (>30 mg/g) in most of the screened excipients.

TABLE 16
Solubility of Compound 1 in various excipients

| Excipient | Solubility (mg/g) |
|---|---|
| Transcutol HP | 118.6 |
| PEG 400 | 77.3 |
| Labrasol ALF | 68.1 |
| Masester E8120 | 65.3 |
| Kolliphor HS 15 | 53.6 |
| Kolliphor RH40 | 52.4 |
| Tween 80 | 49.0 |
| Gelucire 44/14 | 48.9 |
| Tween 20 | 48.6 |
| Gelucire 50/13 | 45.9 |
| Vitamin E TPGS | 40.8 |
| Capryol PGMC | 38.1 |
| Kolliphor ELP | 36.5 |
| Peceol | 22.8 |
| Oleic Acid | 21.0 |
| Maisine CC | 16.7 |
| Lauroglycol FCC | 16.0 |
| Span 80 | 12.2 |
| Labrafil M 1944 CS | 10.7 |
| Labrafil M 2125 CS | 9.3 |
| Miglyol 812N | 2.9 |
| Sesame Oil | 1.9 |

### Example 9: Solubility Testing in Bio-Relevant Media Preparation of FaSSIF

FaSSIF Buffer Solution: 6 mL 0.2 M NaOH, 388.7 mg of NaH₂PO₄, and 608.2 mg of NaCl were dissolved and diluted to 100 mL with water. The pH was 6.51. FaSSIF Media: 34.56 mg of SIF powder was dissolved in 15 mL of FaSSIF buffer solution. The solution was stirred and equilibrated at ambient temperature with light protection for 2 hours. SIF powder was purchased from Biorelevant.com.

### Preparation of FeSSIF

FeSSIF Buffer Solution: 408.4 mg of NaOH, 868.5 mg of acetic acid, and 1.1802 g of NaCl were dissolved and diluted to 100 mL with water. The pH of the solution was 4.98. FeSSIF Media: 168.42 mg of SIF powder was dissolved in 15 mL of FeSSIF buffer solution. The solution was protected from light.

### Preparation of FaSSGF

FaSSGF Buffer Solution: 222 mg of NaCl were dissolved with water, then added 1N HCl adjust the pH to 1.6. FaSSGF Media: 1.2 mg of SIF powder was dissolved in 10 mL of FaSSIF buffer solution. The solution was stirred and equilibrated at ambient temperature with light protection for 2 hours.

### Preparation of FaSSIF-V2

FaSSIF-V2 Buffer Solution: 154.4 mg NaOH, 246.7 mg of maleic acid and 445.6 mg of NaCl were dissolved and diluted to 100 mL with water. The pH was 6.51. FaSSIF-V2 Media: 28.64 mg of FaSSIF-V2 powder (purchased from Biorelevant.com) was dissolved in 8 mL of FaSSIF-V2 buffer solution. The solution was stirred and equilibrated at ambient temperature with light protection for 2 hours.

### Preparation of FeSSIF-V2

FeSSIF-V2 Buffer Solution: 363.3.4 mg of NaOH, 710 mg of maleic acid, and 814.4 mg of NaCl were dissolved and diluted to 100 mL with water. The pH of the solution was 5.8. FeSSIF-V2 Media: 156.16 mg of FeSSIF-V2 powder was dissolved in 8 mL of FeSSIF-V2 buffer solution. The solution was protected from light.

### Solubility Testing in Bio-Relevant Media

Solubility was measured in simulated gastrointestinal fluids (FaSSGF, FaSSIF, FeSSIF, FaSSIF-V2, and FeSSIF-V2) and pH 6.5 buffer, pH 5.8 buffer media at 37° C. for 0.5, 2 and 24 hours About 15 mg of sample was weighed into sample vials and then 3.0 mL of FaSSGF, FaSSIF, FeSSIF, FaSSIF-V2, FeSSIF-V2, pH 6.5 buffer and pH 5.8 buffer media (solubility in buffer solutions without SIF powder was conducted for comparison) was added, respectively. Samples were prepared in duplicate for each medium. The suspensions were shaken at 37° C. for up to 24 hours. At 1, 4 and 24 hours, suspensions were filtered, and the filtrate analyzed by HPLC, and the results presented in Table 17. Residual solids were collected for XRPD analysis, which showed no form change occurred.

TABLE 17
Solubility of Compound 1, Form III in Bio-relevant Media

| | Solubility (mg/mL) | | | pH |
|---|---|---|---|---|
| Media | 1 h | 4 h | 24 h | 24 h |
| FaSSGF, pH 1.6 | | <LOQ | | 1.7 |
| FaSSIF, pH 6.5 | 0.006 | 0.005 | 0.007 | 6.5 |
| FeSSIF, pH 5.0 | 0.073 | 0.075 | 0.090 | 5.0 |
| FaSSIF-V2, pH 6.5 | 0.004 | 0.004 | 0.004 | 6.6 |
| FeSSIF-V2, pH 5.8 | 0.12 | 0.20 | 0.18 | 5.9 |

| | | |
|---|---|---|
| pH 6.5 Buffer | <LOQ | 6.6 |
| pH 5.8 Buffer | <LOQ | 5.8 |

### Example 10: Solubility Testing in Aqueous Media at Various pH

### Preparation of pH 1.0 Solution (0.1 N HCl)

0.833 mL concentrated hydrochloric acid was diluted to 100 mL with water (pH=1.0).

### Preparation of pH 1.2 Buffer Solution

Added appropriate amount of water to 0.1N HCl, adjust the pH to 1.2.

### Preparation of pH 7.4 Buffer Solution

KH₂PO₄ (1360.0 mg) and NaOH (310.0 mg) are mixed in a 200 mL volumetric flask. Water was added to dissolve the solid and dilute to the volume. The flask was shaken to achieve good mixing. (pH=7.42).

### Solubility Testing in Different pH Media and Water

Solubility was measured in pH 1.0 water, pH 1.2 buffer and pH 7.4 buffer at room temperature and 37° C. About 10-15 mg of sample was weighed into sample vials and then 2.0/3.0 mL of 0.1 N HCl solution, pH 1.2 buffer and pH 7.4 buffer media, and water were added, respectively. Samples were run in duplicate for each medium. The suspensions were shaken at room temperature or 37° C. At 4, 24, 48 hours, suspensions were centrifuged, and the filtrate analyzed by HPLC; results presented in Table 18). Residual solid was collected for XRPD analysis, though no form change occurred during testing. In all instances, very low solubility was observed.

TABLE 18
Solubility in Aqueous Media at Various pH

| | | | Solubility (mg/mL) | | |
|---|---|---|---|---|---|
| Media | Temp | pH | 4 h | 24 h | 48 h |
| pH 1.0 (0.1N HCl) | RT | 1.0 | 0.0007 | 0.0008 | 0.0016 |
| pH 7.4 Buffer | RT | 7.4 | N/A | 0.0008 | 0.0017 |
| pH 1.2 Buffer | 37° C. | 1.2 | 0.0009 | 0.0009 | N/A |
| pH 7.4 Buffer | 37° C. | 7.4 | 0.0008 | 0.0008 | N/A |
| Water (pH 5.0) | 37° C. | 5.0 | 0.0006 | 0.0007 | N/A |

### Example 11: Single Crystal X-Ray Structure of Compound 1, Form III

Single crystal X-ray diffraction studies were carried out on a Bruker Smart APEX II CCD diffractometer equipped with Cu K_{α} radiation (λ=1.54178 Å).

Crystals of Compound 1, Form III were grown from EtOAc/Pentane.

A 0.23×0.2×0.17 mm piece of a colorless crystal was mounted on a Cryoloop with Paratone oil. Data were collected in a nitrogen gas stream at 100(2) K using ϕ and ω scans. Crystal-to-detector distance was 40 mm and exposure time was 1, 2, 3, or 5 seconds depending on the 2θ range per frame using a scan width of 1.25°. Data collection was 97.7% complete to 67.679° in θ. A total of 27594 reflections were collected covering the indices, −11<=h<=11, −11<=k<=11, −13<=1<=13. 6852 reflections were found to be symmetry independent, with a Rᵢₙₜ of 0.0232. Indexing and unit cell refinement indicated a Triclinic lattice. The space group was found to be P1. The data were integrated using the Bruker SAINT Software program and scaled using the SADABS software program. Solution by direct methods (SHELXT) produced a complete phasing model consistent with the proposed structure.

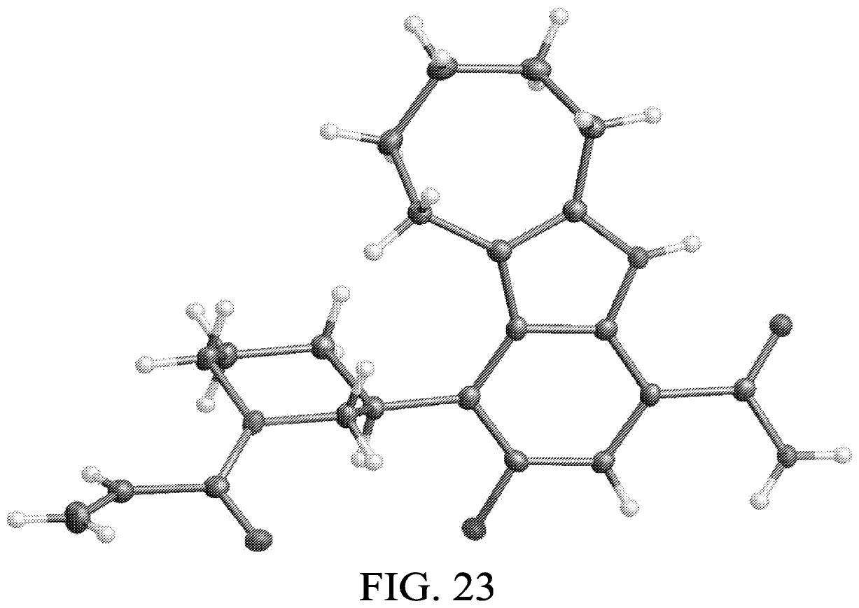

All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. Absolute stereochemistry was conclusively assigned (Flack=0.04(3)). The structure is shown in FIG. 23. There are two copies of the compound in the asymmetric unit. Crystallographic data were as follows:

| Crystal system | Triclinic | | |
|---|---|---|---|
| Space group | P1 | | |
| Unit cell dimensions | a = 9.61280(10) Å | | α = 110.7050(10)° |
| | b = 9.78860(10) Å | | β = 90.9170(10)° |
| | c = 10.85140(10) Å | | γ = 96.4060(10)° |
| Volume | 947.470(17) Å³ | | |
| Z | 2 | | |
| Density (calculated) | 1.344 | Mg/m³ | |
| Absorption coefficient | 0.764 | mm⁻¹ | |

| | |
|---|---|
| F(000) | 408 |
| Crystal size | 0.23 × 0.2 × 0.17 mm³ |
| Theta range for data collection | 4.363 to 70.453° |
| Index ranges | −11 <= h <= 11, −11 <= k <= 11, −13 <= l <= 13 |
| Reflections collected | 27594 |
| Independent reflections | 6852 [R(int) = 0.0232] |
| Completeness to theta = 67.679° | 97.7% |
| Absorption correction | Semi-empirical from equivalents |
| Max. and min. transmission | 0.5220 and 0.4322 |
| Refinement method | Full-matrix least-squares on F² |
| Data/restraints/parameters | 6852/3/505 |
| Goodness-of-fit on F² | 1.026 |
| Final R indices [I > 2sigma(I)] | R1 = 0.0262, wR2 = 0.0715 |
| R indices (all data) | R1 = 0.0266, wR2 = 0.0720 |
| Absolute structure parameter | 0.04(3) |
| Largest diff. peak and hole | 0.181 and −0.168 e. Å⁻³ |

### Formulation Development

Formulation development and batch manufacture of Compound 1 softgel capsules was investigated. Compound 1, Form III was used as starting material. Seven formulations were prepared, and their solubility, kinetic solubility, permeability, physical stability and chemical stability evaluated. Formulation A7 comprises Compound 1, PEG 400, Lauroglycol 90, Vitamin E TPGS and BHT. Formulation A7 softgel capsules were manufactured at three strengths (2 mg, 5 mg & 25 mg). The softgel shell comprises Gelatin (Type 195), Sorbitol Special-Glycerin Blend, Titanium Dioxide, FD&C Blue #1, Red Iron Oxide and Purified Water.

### Example 12: Preparation of Formulations A1-A6

Six formulations (A1, A2, A3, A4, A5 and A6) were prepared with a target Compound 1 concentration of 22.7 mg/g. These formulations were designed to create a variety of formulation types, based on ratios of glycerides, surfactants, and hydrophilic cosolvents. Butylated Hydroxytoluene (BHT) was included in each of the formulation as an antioxidant to minimize any potential oxidation degradation. Table 19 shows the formulation compositions.

TABLE 19
Formulations A1-A6

| Components | % w/w | mg/g |
|---|---|---|
| Formulation A1 | | |
| Compound 1 | 2.27% | 22.7 |
| PEG 400 | 30.00% | 300 |
| Labrasol ALF | 44.00% | 440 |
| Vitamin E TPGS | 23.71% | 237.1 |
| BHT | 0.02% | 0.2 |
| Formulation A2 | | |
| Compound 1 | 2.27% | 22.7 |
| Oleic Acid | 65.00% | 650 |
| Transcutol HP | 12.00% | 120 |
| Tween 20 | 20.71% | 207.1 |
| BHT | 0.02% | 0.2 |
| Formulation A3 | | |
| Compound 1 | 2.27% | 22.7 |
| Masester E8120 | 20.00% | 200 |
| Gelucire 44/14 | 39.00% | 390 |
| Kolliphor HS 15 | 38.71% | 387.1 |
| BHT | 0.02% | 0.2 |
| Formulation A4 | | |
| Compound 1 | 2.27% | 22.7 |
| PEG 400 | 30.00% | 300 |
| Lauroglycol FCC | 20.00% | 200 |
| Vitamin E TPGS | 47.71% | 477.1 |
| BHT | 0.02% | 0.2 |
| Formulation A5 | | |
| Compound 1 | 2.27% | 22.7 |
| Peceol | 48.71% | 487.1 |
| Masester E8120 | 49.00% | 490 |
| BHT | 0.02% | 0.2 |
| Formulation A6 | | |
| Compound 1 | 2.27% | 22.7 |
| Maisine CC | 37.71% | 377.1 |
| Capryol PGMC | 30.00% | 300 |
| Gelucire 44/14 | 30.00% | 300 |
| BHT | 0.02% | 0.2 |

### Example 13: Solubility of Formulations A1-A6

The solubility of formulations A1-A6 was evaluated via fiber optic dissolution using a Pion Rainbow Dynamic Dissolution Monitor with Fiber Optic and Distek Dissolution System 2500 (Dissolution Apparatus Type II). Three biorelevant media were evaluated: FaSSGF (pH=1.3), FaSSIF (pH=6.5), and FeSSIF (pH=5.5), as detailed in Table 20.

TABLE 20
Biorelevant Media Composition

| | Concentration (mM) | | |
|---|---|---|---|
| | FaSSIF Media | FeSSIF Media | FaSSGF Media |
| Component | (pH 6.5) | (pH 5.5) | (pH 1.3) |
| Sodium Taurocholate | 3.0 | 15.0 | 0.08 |
| Lecithin | 0.75 | 3.75 | 0.02 |
| Sodium Chloride | 106 | 203 | 34.2 |
| Monobasic Sodium | 28.4 | 0 | 0 |
| Phosphate | | | |
| Sodium Hydroxide | 8.7 | 101 | 0 |
| Acetic Acid | 0 | 144 | 0 |
| Hydrochloric Acid | 0 | 0 | 25.1 |

The solubility of each formulation was monitored continuously in each dissolution vessel (N=1, 500 mL volume, 37° C., 75 rpm paddle speed) over a 6-hour period by fiber optic probes. All formulations showed increased solubility over crystalline Compound 1 alone (used as control) in each media, with A1, A3 and A4 exhibiting the highest solubility.

### Example 14: Permeability of Formulations A1, A3 and A4

The permeability of formulations A1, A3 and A4 was evaluated using a Pion pFlux apparatus (2 compartments) and Pion Rainbow Dynamic Dissolution Monitor with fiber optic probes. Test formulation was added to the compartment containing FaSSIF media and Acceptor Sink Buffer was placed in the second compartment. The two compartments were separated by a membrane filter coated with GIT-0 Lipid Solution. (This setup mimics passive diffusion in the intestinal membrane). A fiber optic probe in each compartment continuously monitored Compound 1 concentration on each side of the membrane, over a 6-hour period. Table 21 shows the percentage Compound 1 permeability for each formulation after 6 hours.

TABLE 21
Permeability of formulations A1, A3 and A4 after 6 hours

| Formulation | % permeated (6 hours) |
|---|---|
| A1 | 16.5% |
| A3 | 12.9% |
| A4 | 20.4% |

### Example 15: Physical Stability Evaluation (Temperature Hold at Rt and 40° C.)

The physical stability of formulations A1, A3 and A4 was evaluated at room temperature (15-25° C.) and 40° C. Two sets of samples (˜3 g each) were aliquoted into single glass vials. One set was held at room temperature for 7 days and the other at 40° C., for 7 days. Changes in appearance such as color change, phase separation or precipitation were monitored through visual observation. No signs of physical instability at room temperature or 40° C. were observed.

### Example 16: Physical Stability Evaluation (Temperature Cycled ˜40° C./−20° C.)

The physical stability of formulations A1, A3 and A4 was evaluated under temperature cycling conditions. A sample (˜3 g each) of each test formulation (A1, A3 and A4) was aliquoted into a glass vial and exposed to three cycles of high (40° C.) and low (˜20° C.) temperatures for ˜24 hours at each temperature condition. As shown in Table 22, no color change, phase separation or precipitation was observed.

TABLE 22
Temperature Cycling of Formulations A1, A3 and A4

| Time Point | A1 | A3 | A4 |
|---|---|---|---|
| Initial | Clear yellow solution | Clear yellow solution | Clear yellow solution |
| Cycle 1 (40° C.) | Clear yellow solution. | Clear yellow solution. | Clear yellow solution. |
| | No phase separation. No | No phase separation. No | No phase separation. No |
| | precipitation. | precipitation. | precipitation. |
| Cycle 1 (−20° C.) | Opaque yellow semisolid | Opaque yellow semisolid | Opaque yellow semisolid |
| Cycle 2 (40° C.) | Clear yellow solution. | Clear yellow solution. | Clear yellow solution. |
| | No phase separation. No | No phase separation. No | No phase separation. No |
| | precipitation. | precipitation. | precipitation. |
| Cycle 2 (−20° C.) | Opaque yellow semisolid | Opaque yellow semisolid | Opaque yellow semisolid |
| Cycle 3 (40° C.) | Clear yellow solution. | Clear yellow solution. | Clear yellow solution. |
| | No phase separation. No | No phase separation. No | No phase separation. No |
| | precipitation. | precipitation. | precipitation. |

### Example 17: Plasticizer Challenge

The plasticizer challenge evaluates the possible effect of plasticizer migration into the fill solution after encapsulation within a softgel capsule. Two plasticizers were evaluated, Sorbitol Sorbitan Solution and Sorbitol Special-Glycerin Blend A810. The challenge was performed with 5% of each plasticizer spiked into each formulation. The spiked samples were stored at room temperature (15-25° C.) and 40° C. and observations noted at T=O and then daily for up to 7 days. No precipitation was observed. Phase separation was observed as clear droplets on the bottom of the vials in all of the formulations in the presence of both plasticizers, suggesting the plasticizer and fill migration are not miscible and the plasticizer unlikely to migrate from the capsule shell. Tables 23 and 24 summarize the results.

TABLE 23
Plasticizer Challenge with Sorbitol Sorbitan Solution

| | | | |
|---|---|---|---|
| Time Point | A1 | A3 | A4 |

Room Temp

| | | | |
|---|---|---|---|
| Initial | Clear yellow solution | Clear yellow solution | Clear yellow solution |
| Day 1 | Opaque yellow | Opaque yellow | Opaque yellow |
| | semisolid | semisolid | semisolid |
| Day 2 | Opaque yellow | Opaque yellow | Opaque yellow |
| | semisolid | semisolid | semisolid |
| Day 5 | Opaque yellow | Opaque yellow | Opaque yellow |
| | semisolid | semisolid | semisolid |
| Day 6 | Opaque yellow | Opaque yellow | Opaque yellow |
| | semisolid | semisolid | semisolid |
| Day 7 | Opaque yellow | Opaque yellow | Opaque yellow |
| | semisolid | semisolid | semisolid |

40° C.

| | | | |
|---|---|---|---|
| Initial | Clear yellow solution | Clear yellow solution | Clear yellow solution |
| Day 1 | Clear yellow solution | Hazy yellow solution | Hazy yellow solution |
| | with droplets on | with droplets on the | with droplets on the |
| | bottom of vial | bottom of vial | bottom of vial |
| Day 2 | Slightly hazy yellow | Hazy yellow solution | Hazy yellow solution |
| | solution with droplets | with droplets at bottom | with droplets at bottom |
| | at bottom of vial | of vial | of vial |
| Day 5 | Slightly hazy yellow | Hazy yellow solution | Hazy yellow solution |
| | solution with droplets | with droplets at bottom | with droplets at bottom |
| | at bottom of vial | of vial | of vial |
| Day 6 | Clear yellow solution | Hazy yellow solution | Hazy yellow solution |
| | with droplets on | with droplets at bottom | with droplets at bottom |
| | bottom of vial | of vial | of vial |
| Day 7 | Clear yellow solution | Hazy yellow solution | Hazy yellow solution |
| | with droplets on | with droplets at bottom | with droplets at bottom |
| | bottom of vial | of vial | of vial |

TABLE 24
Plasticizer Challenge with Sorbitol Special - Glycerin Blend

| | | | |
|---|---|---|---|
| Time Point | A1 | A3 | A4 |

Room Temp

| | | | |
|---|---|---|---|
| Initial | Clear yellow solution | Clear yellow solution | Clear yellow solution |
| Day 1 | Opaque yellow | Opaque yellow | Opaque yellow |
| | semisolid | semisolid | semisolid |
| Day 2 | Opaque yellow | Opaque yellow | Opaque yellow |
| | semisolid | semisolid | semisolid |
| Day 5 | Opaque yellow | Opaque yellow | Opaque yellow |
| | semisolid | semisolid | semisolid |
| Day 6 | Opaque yellow | Opaque yellow | Opaque yellow |
| | semisolid | semisolid | semisolid |
| Day 7 | Opaque yellow | Opaque yellow | Opaque yellow |
| | semisolid | semisolid | semisolid |

40° C.

| | | | |
|---|---|---|---|
| Initial | Clear yellow solution | Clear yellow solution | Clear yellow solution |
| Day 1 | Slightly hazy yellow | Hazy yellow solution | Hazy yellow solution |
| | solution | | |
| Day 2 | Slightly hazy yellow | Hazy yellow solution | Hazy yellow solution |
| | solution with droplets | with droplets at bottom | with droplets at bottom |
| | at bottom of vial | of vial | of vial |
| Day 5 | Slightly hazy yellow | Hazy yellow solution | Hazy yellow solution |
| | solution with droplets | with droplets at bottom | with droplets at bottom |
| | at bottom of vial | of vial | of vial |
| Day 6 | Slightly hazy yellow | Hazy yellow solution | Hazy yellow solution |
| | solution with droplets | with droplets at bottom | with droplets at bottom |
| | at bottom of vial | of vial | of vial |
| Day 7 | Slightly hazy yellow | Hazy yellow solution | Hazy yellow solution |
| | solution with droplets | with droplets at bottom | with droplets at bottom |
| | at bottom of vial | of vial | of vial |

### Example 18: Formulation A7

Formulation A7 was prepared according to the same conditions as formulations A1-A6. Formulation A7 contains the same components and amounts as formulation A4, other than Lauroglycol 90 (Propylene Glycol Monolaurate (Type II)) was used in place of Lauroglycol FCC (Propylene Glycol Monolaurate (Type I)). These excipients contain different ratios of mono- and di-esters.

| Formulation A7 | % w/w | mg/g |
|---|---|---|
| Compound 1 | 2.27% | 22.7 |
| PEG 400 | 30.00% | 300 |
| Lauroglycol 90 | 20.00% | 200 |
| Vitamin E TPGS | 47.71% | 477.1 |
| BHT | 0.02% | 0.2 |

### Example 19: Maximum Drug Solubility

The maximum solubility of Compound 1 in formulations A1, A3, A4 and A7 was evaluated to determine the maximum possible drug load per formulation.

A placebo formulation of each prototype was prepared and super-saturated with Compound 1. The mixtures were incubated on a temperature-controlled shaker with glass mixing beads for at least 48 hours at 40° C. to ensure maximum solubility was achieved. The samples were then centrifuged using 0.45 μm PVDF filter to separate the liquid and solid portions. The amount of dissolved Compound 1 in the filtrate was quantified by HPLC analysis and shown in Table 25.

TABLE 25
Compound 1 solubility in formulations A1, A3, A4 and A7

| Formulation | Solubility (mg/g) |
|---|---|
| A1 | 74.7 |
| A3 | 56.9 |
| A4 | 59.2 |
| A7 | 63.1 |

### Example 20: Stability Testing

Formulations A1, A3 and A7 were prepared and divided into four sets of samples, to which were added:
Nothing (control)
5% Water
Gel 1: Gel 004007-L3DXHBHM contains Sorbitol Special-Glycerin Blend as plasticizer
Gel 2: Gel 004013-LSMHRS1HM contains Sorbitol Sorbitan Solution as plasticizer

Both gels were prepared using Titanium Dioxide, Iron Oxide, Red and FD&C Blue #1 as colorants. One dried swatch of gel 004007 was added to the gel 1 samples and one dried swatch of 004013 was added to gel 2.

The samples were stored in 6 mL amber glass vials at: room temperature, 40° C., 50° C. and 5° C. for up 8 weeks. Control samples (Compound 1 alone) were analyzed in parallel with the study samples and control peaks were subtracted from the total impurities. Compound 1 related impurities, after storage at 40° C., are listed in Table 26.

TABLE 26
Compound 1 Related Impurities (% adjusted area) at 40°
C. (RRT = Relative Retention Time)

| RRT | T = 0 | T = 3 weeks | T = 8 weeks |
|---|---|---|---|
| 0.47 | — | — | 0.07 |
| 0.73 | 0.05 | 0.06 | 0.05 |
| 0.79 | <LOQ | <LOQ | <LOQ |
| 0.84 | 0.07 | 0.05 | 0.06 |
| 0.91 | 0.10 | 0.07 | 0.08 |
| 0.93 | 0.06 | 0.07 | 0.05 |
| 1.04 | — | — | 0.12 |
| 1.08 | 0.07 | 0.07 | 0.06 |
| 1.13 | 0.36 | 0.30 | 0.32 |
| Total | 0.71 | 0.62 | 0.81 |

The stability results are presented in Tables 27-34. Most of the impurities observed at T=0 were present in the Compound 1 alone starting material and thus not considered as formulation related impurities.

TABLE 27
A1, A3, A7 Formulation Stability at 40° C., after 0, 1, 2 and 8
weeks; and at 5° C. and 50° C., after 2 weeks

| | Purity | | | | | |
|---|---|---|---|---|---|---|
| | Temp: | | | | | |
| | 40° C. | 40° C. | 40° C. | 40° C. | 5° C. | 50° C. |
| | Week: | | | | | |
| | 0 | 1 | 2 | 8 | 2 | 2 |
| Al alone | 99.4 | 96.9 | 97.5 | — | 96.1 | 96.9 |
| A1 + Gel 1 | 99.4 | 95.4 | 95.3 | — | 98.3 | 98.6 |
| A1 + Gel 2 | 99.4 | 96.5 | 95.6 | — | 98.7 | 99.1 |
| A1 + Water | 102.9 | 98.1 | 99.3 | — | 102.0 | 102.2 |
| A3 alone | 99.6 | 101.3 | 95.4 | — | 98.9 | 100.0 |
| A3 + Gel 1 | 99.6 | 96.1 | 92.6 | — | 98.8 | 98.0 |
| A3 + Gel 2 | 99.6 | 95.8 | 99.4 | — | 99.9 | 100.0 |
| A3 + Water | 99.7 | 100.4 | 103.9 | — | 103.5 | 102.2 |
| A7 alone | 99.2 | 96.0 | 99.9 | 102.7 | 101.6 | 100.3 |
| A7 + Gel 1 | 99.2 | 96.0 | 98.6 | 99.7 | 99.1 | 98.6 |
| A7 + Gel 2 | 99.2 | 95.3 | 99.2 | 100.9 | 100.7 | 99.0 |
| A7 + Water | 99.6 | 100.9 | 102.7 | 98.9 | 103.0 | 102.1 |

TABLE 28
Formulation Al: Stability (% adjusted area) +/− Water at 40° C.

| | A1 | | | A1 + H₂O | | |
|---|---|---|---|---|---|---|
| | Week: | | | | | |
| | 0 | 1 | 2 | 0 | 1 | 2 |
| | Temp: | | | | | |
| | 40° C. | 40° C. | 40° C. | 40° C. | 40° C. | 40° C. |
| Non-Cpd1 | 0.00 | 0.18 | 0.19 | 0.07 | 0.20 | 0.25 |
| Cpd1 | 0.54 | 0.71 | 0.95 | 0.55 | 0.74 | 0.77 |
| TOTAL | 0.54 | 0.89 | 1.14 | 0.62 | 0.94 | 1 |

TABLE 29
Stability Formulation A1 (% adjusted area) +/−
Water at 5° C. and 50° C.

| | |
|---|---|
| A 1 | A1 + H₂O |

| Week: | 2 | 2 | 2 | 2 |
|---|---|---|---|---|
| Temp: | 5° C. | 5° C. | 5° C. | 5° C. |
| Non- Cpd1 | 0.14 | 0.31 | 0.00 | 0.09 |
| Cpd1 | 0.88 | 0.89 | 0.76 | 0.72 |
| TOTAL | 1.02 | 1.20 | 0.76 | 0.79 |

TABLE 30
Formulation A3 Stability (% adjusted area) +/− Water at 40° C.

| | A3 | | | A3 + H₂O | | |
|---|---|---|---|---|---|---|
| | Week: | | | | | |
| | 0 | 1 | 2 | 0 | 1 | 2 |
| | Temp: | | | | | |
| | 40° C. | 40° C. | 40° C. | 40° C. | 40° C. | 40° C. |
| Non- Cpd1 | 0.07 | 0.00 | 0.00 | 0.07 | 0.06 | 0.00 |
| API Cpd1 | 0.55 | 0.64 | 0.79 | 0.54 | 0.72 | 0.73 |
| TOTAL | | | | | | |

TABLE 31
Formulation A3 Stability (% adjusted area) +/−
Water at 5° C. and 50° C.

| | |
|---|---|
| A 3 | A3 + H₂O |

| Week: | 2 | 2 | 2 | 2 |
|---|---|---|---|---|
| Temp: | 5° C. | 50° C. | 5° C. | 50° C. |
| Non- Cpd1 | 0.00 | 0.00 | 0.00 | 0.00 |
| Cpd1 | 0.63 | 0.80 | 0.58 | 0.86 |
| TOTAL | 0.63 | 0.80 | 0.58 | 0.86 |

TABLE 32
Formulation A7 Stability (% adjusted area) +/− Water at 40° C.

| | A 7 | | | | A7 + H₂O | | | |
|---|---|---|---|---|---|---|---|---|
| | Temp: | | | | | | | |
| | 40° C. | 40° C. | 40° C. | 40° C. | 40° C. | 40° C. | 40° C. | 40° C. |
| | Week: | | | | | | | |
| | 0 | 1 | 2 | 8 | 0 | 1 | 2 | 8 |
| Non- Cpd1: | 0.00 | 0.05 | 0.00 | 0.13 | 0.00 | 0.00 | 0.00 | 0.07 |
| Cpd1 | 0.54 | 0.73 | 0.71 | 0.71 | 0.54 | 0.7 | 0.71 | 0.83 |
| TOTAL | 0.54 | 0.78 | 0.71 | 0.84 | 0.54 | 0.7 | 0.71 | 0.9 |

TABLE 33
Formulation A7 Stability (% adjusted area) +/−
Water at 5° C. and 50° C.

| | |
|---|---|
| A7 | A7 + H₂O |

| Week: | 2 | 2 | 2 | 2 |
|---|---|---|---|---|
| Temp: | 5° C. | 50° C. | 5° C. | 50° C. |
| Non- Cpd1: | 0.00 | 0.09 | 0.00 | 0.00 |
| Cpd1 | 0.70 | 0.77 | 0.72 | 0.63 |
| TOTAL | 0.70 | 0.86 | 0.72 | 0.63 |

TABLE 34
A1, A3, A7 Formulation Stability +/− Gel or Gel 2 at 40° C., after 0,
1, 2 and 8 weeks; and at 5° C. and 50° C., after 2 weeks

| | Week: | | | | | | | | | | | |
|---|---|---|---|---|---|---|---|---|---|---|---|---|
| | 0 | 1 | 2 | 8 | 2 | 2 | 0 | 1 | 2 | 8 | 2 | 2 |
| | Temp: | | | | | | | | | | | |
| | 40° C. | 40° C. | 40° C. | 40° C. | 5° C. | 50° C. | 40° C. | 40° C. | 40° C. | 40° C. | 5° C. | 50° C. |
| | A1 + Gel 1 | | | | | | A1 + Gel 2 | | | | | |
| Non- Cpd1 | 0.00 | 0.18 | 0.12 | — | 0.00 | 0.00 | 0.00 | 0.23 | 0.14 | — | 0.00 | 0.00 |
| Cpd1 | 0.54 | 0.7 | 0.87 | — | 0.76 | 0.74 | 0.54 | 0.71 | 0.89 | — | 0.76 | 0.81 |
| TOTAL | 0.54 | 0.25 | 0.99 | — | 0.76 | 0.74 | 0.54 | 0.94 | 1.03 | — | 0.76 | 0.81 |
| | A3 + Gel 1 | | | | | | A3 + Gel 2 | | | | | |
| Non- Cpd1 | 0.07 | 0.07 | 0.00 | — | 0.00 | 0.00 | 0.00 | 0.05 | 0.06 | — | 0.00 | 0.00 |
| Cpd1 | 0.55 | 0.69 | 0.83 | — | 0.6 | 0.84 | 0.55 | 0.74 | 0.78 | — | 0.6 | 0.85 |
| TOTAL | 0.62 | 0.76 | 0.83 | — | 0.6 | 0.84 | 0.55 | 0.79 | 0.84 | — | 0.6 | 0.85 |
| | A7 + Gel 1 | | | | | | A7 + Gel 2 | | | | | |
| Non- Cpd1 | 0.00 | 0.05 | 0.00 | 0.00 | 0.00 | 0.00 | 0.00 | 0.00 | 0.00 | 0.00 | 0.00 | 0.00 |
| Cpd1 | 0.54 | 0.73 | 0.69 | 0.76 | 0.69 | 0.65 | 0.54 | 0.74 | 0.71 | 0.79 | 0.69 | 0.61 |
| TOTAL | 0.54 | 0.78 | 0.69 | 0.76 | 0.69 | 0.65 | 0.54 | 0.74 | 0.71 | 0.79 | 0.69 | 0.61 |

All three formulations (A1, A3, and A7) showed similar stability up to two weeks at 40° C., 50° C. and 5° C. After 8-weeks, the stability of A7 stability 40° C. was analyzed.

### Example 21: Batch Manufacture—2 mg, 5 mg & 25 mg Capsules, Based on A7

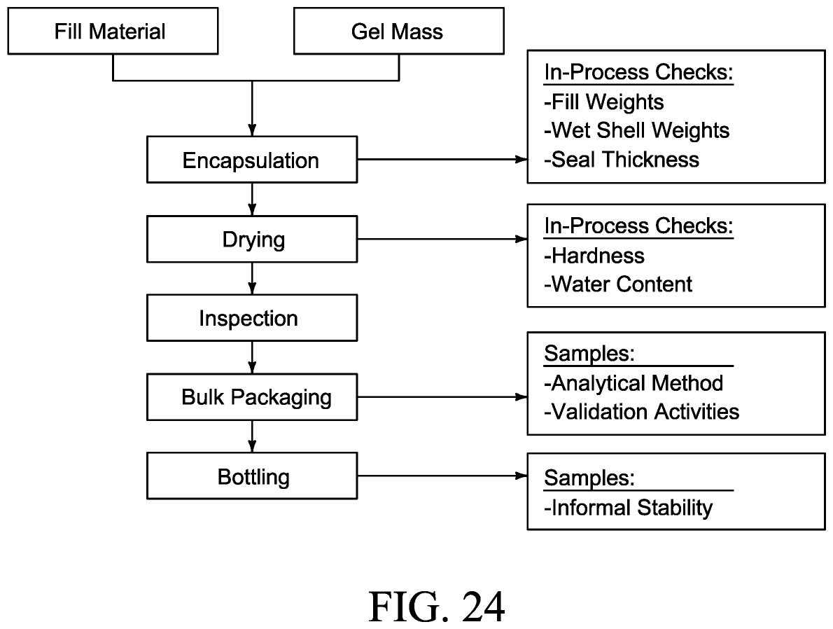

FIG. 24 represents a flow diagram of the batch manufacturing process. Three strengths of softgel capsules were manufactured, containing 2 mg, 5 mg and 25 mg of Compound 1. The composition of the fill formulations was equivalent, and the dose adjusted by varying the capsule size. The fill mix was split for the 5 mg and 2 mg batches. The composition of the fill formulations is presented in Table 35. The composition of the softgel shell is shown in Table 36.

TABLE 35
Composition of 2 mg, 5 mg and 25 mg Capsules (Theoretical
Lot quantity = 2,270 capsules for each dose)

| | | Fill Component | mg/Softgel | g/Batch |
|---|---|---|---|---|
| 2 | mg | Compound 1 | 2.00 | 27.24 |
| | | Polyethylene Glycol 400, NF, EP (Macrogols Type-400) | 26.40 | 360.000 |
| | | Propylene Glycol Monolaurate, NF, EP (Type II) | 17.60 | 240.000 |
| | | Vitamin E TPGS, NF | 41.98 | 572.52 |
| | | Butylated Hydroxytoluene (BHT), NF | 0.02 | 0.240 |

| | | | | |
|---|---|---|---|---|
| TOTAL | 88.00 | mg | 1,200.00 | g |

| | | | | |
|---|---|---|---|---|
| 5 | mg | Compound 1 | 5.00 | 27.24 |
| | | Polyethylene Glycol 400, NF, EP (Macrogols Type-400) | 66.00 | 360.000 |
| | | Propylene Glycol Monolaurate, NF, EP (Type II) | 44.00 | 240.000 |
| | | Vitamin E TPGS, NF | 104.96 | 572.52 |
| | | Butylated Hydroxytoluene (BHT), NF | 0.04 | 0.240 |

| | | | | |
|---|---|---|---|---|
| TOTAL | 220.00 | mg | 1,200.00 | g |

| | | | | |
|---|---|---|---|---|
| 25 | mg | Compound 1 | 25.00 | 56.825 |

| Polyethylene Glycol 400, NF, EP (Macrogols Type-400) | 330.00 | 750.000 | |
|---|---|---|---|
| Propylene Glycol Monolaurate, NF, EP (Type II) | 220.00 | 500.000 | |
| Vitamin E TPGS, NF | 524.78 | 1192.675 | |
| Butylated Hydroxytoluene (BHT), NF | 0.22 | 0.500 | |
| TOTAL | 1,100.00 | 2,500.00 | g |

TABLE 36
Softgel Shell Components

| | Calculated amount per softgel (mg) | | |
|---|---|---|---|
| | (per dry bases) | | |
| Gel Component | 25 mg | 5 mg | 2 mg |
| Gelatin, (Type 195) NF, EP (Tested to JP) | 292.510 | 92.847 | 54.640 |
| Sorbitol Special, NF, EP - Glycerin, USP Blend | 202.667 | 64.330 | 37.858 |
| Titanium dioxide, USP, EP | 1.393 | 0.442 | 0.260 |
| FD&C Blue #1 | 0.174 | 0.055 | 0.033 |
| Iron Oxide, Red | 0.052 | 0.017 | 0.010 |

The fill mixtures were prepared using a Becomix 2.5 L mixing vessel. The vessel was preheated to 40° C. Propylene Glycol Monolaurate, Vitamin E TPGS, and BHT were added to the Becomix vessel and mixed at 1.0 m/s agitator speed (range 0.5-1.5 m/s in Right mode) and homogenizer speed at 5.0 m/s (range 5-10 m/s) for NLT 15 under vacuum maintained at −0.9 bar. The product temperature was maintained between 38° C.-42° C. throughout mixing process by adjusting agitator and homogenizing speed as needed within their respective validated range. Compound 1 was then added under yellow light turned on to protect from light. The Compound 1 container was rinsed with the Polyethylene Glycol 400 to ensure complete transfer. The mixture was mixed for another 60 minutes at 40° C. The solution was then de-aerated for 30 minutes at 40° C. and filtered through a 325-mesh in-line filter on discharge from the Becomix vessel to remove any undissolved material. An in-process assay was performed on the fill material and target fill weights were adjusted accordingly (Table 37). The fill material was stored in a stainless-steel hopper at 40° C. until ready for encapsulation.

TABLE 37
In-Process Assay Results and Fill Weight Adjustments

| | | In-Process Assay | Theoretical Fill | Adjusted Fill |
|---|---|---|---|---|
| Compound 1 | | (% LC) | Weight | Weight |
| 2 | mg | 98.6 | 0.088 g | 0.090 g |
| 5 | mg | | 0.220 g | 0.223 g |
| 25 | mg | 92.7 | 1.100 g | 1.185 g |

A heated closed head hopper was used during encapsulation at 40° C. Three dies were evaluated for the three capsule strengths; all three dies produced suitable seals.
G2VD was used for the 2 mg
G4VH was used for the 5 mg
G20BA was used for the 25 mg

The gel mass was prepared using gelatin, Sorbitol Special-Glycerin Blend A810 as plasticizer, and purified water and was color converted to opaque blue color by adding Titanium dioxide, red iron oxide, FD&C Blue #1, and purified water. The in-process fill weight, shell weights and seal thickness were monitored every 10 min throughout the run. A summary of the in-process checks is presented in Table 38.

TABLE 38
In-process Checks

| Cmpd 1 | Test | Acceptance Criteria | Results |
|---|---|---|---|
| 2 mg | Shell | Target: 0.133 g | Average: 0.137 g |
| | Weights | Range: 0.122-0.144 g | Range: 0.135-0.138 g (n = 7) |
| | Fill | Target: 0.090 g | Average: 0.090 g |
| | Weights | Range: 0.088-0.093 g | Range: 0.089-0.091 g (n = 7) |
| | Seal | Action Limit: | Leading Seal: |
| | Thickness | <0.012 inch | Average: 0.018 in. |
| | | Minimum: 0.010 inch | Range: 0.016-0.022 |
| | | | inch (n = 7) |
| | | | Trailing Seal: |
| | | | Average: 0.014 in. |
| | | | Range: 0.012-0.015 |
| | | | inch (n = 7) |
| 5 mg | Shell | Target: 0.226 g | Average: 0.219 g |
| | Weights | Range: 0.208-0.244 g | Range: 0.214-0.222 g (n = 4) |
| | Fill | Target: 0.223 g | Average: 0.224 g |
| | Weights | Range: 0.216-0.230 g | Range: 0.220-0.227 g (n = 4) |
| | Seal | Action Limit: | Leading Seal: |
| | Thickness | <0.012 inch | Average: 0.017 in. |
| | | Minimum: 0.010 inch | Range: 0.015-0.018 |
| | | | inch (n = 4) |
| | | | Trailing Seal: |
| | | | Average: 0.013 in. |
| | | | Range: 0.012-0.013 |
| | | | inch (n = 4) |
| 25 mg | Shell | Target: 0.712 g | Average: 0.680 g |
| | Weights | Range: 0.655-0.769 g | Range: 0.655-0.690 g (n = 5) |
| | Fill | Target: 1.185 g | Average: 1.193 g |
| | Weights | Range: 1.149-1.220 g | Range: 1.188-1.196 g (n = 5) |
| | Seal | Action Limit: | Leading Seal: |
| | Thickness | <0.012 inch | Average: 0.019 in. |
| | | Minimum: 0.010 inch | Range: 0.017-0.022 |
| | | | inch (n = 5) |
| | | | Trailing Seal: |
| | | | Average: 0.016 in. |
| | | | Range: 0.014-0.018 |
| | | | inch (n = 5) |

### Example 22: Drying and Moisture Content

Drying of the filled capsules was monitored by measuring fill moisture and hardness over several days. The capsules were dried to a hardness within the range:
25 mg capsules: 7.0-10.0 Newton
2 mg & 5 mg capsules: 7.0-11.0 Newton

At each time point, five capsules were tested for hardness using Bareiss Hardness Durometer. A capsule was placed on the test plate with seams parallel to the plate. The durometer plunger was adjusted until just in contact with the capsule. The hardness test measures the applied force necessary for a 2 mm displacement of the softgel. Moisture content (FM) of the filled capsules was also tested using a Karl Fischer Titrator. The fill material from five capsules was removed and tested in duplicate. The hardness and moisture results are presented in Table 39.

TABLE 39
Capsules hardness and Fill Moisture Content
for 2 mg, 5 mg and 25 mg Capsules

| Capsule | Day | Av hardness (N), n = 5 | Av moisture (% Water), n = 2 |
|---|---|---|---|
| 2 mg | 1 | 4.26 | 8.6809 |
| | 2 | 6.18 | 6.6384 |
| | 3 | 7.14 | 5.7272 |
| | 4 | 9.22 | 4.5799 |
| | 5 | 9.88 | 4.1273 |
| | 6 | 10.78 | 3.5853 |
| 5 mg | 1 | 3.92 | 7.5853 |
| | 2 | 5.54 | 6.2152 |
| | 3 | 6.46 | 5.4281 |
| | 5 | 8.40 | 4.5577 |
| | 6 | 9.16 | 3.9948 |
| | 7 | 9.96 | 4.6159 |
| | 8 | 10.34 | 3.7424 |
| | 9 | 9.52 | 3.7211 |
| 25 mg | 1 | 4.10 | 6.2661 |
| | 2 | 5.28 | 5.4890 |
| | 4 | 6.74 | 4.7533 |
| | 6 | 7.86 | 4.3067 |
| | 7 | 8.44 | 4.1007 |
| | 8 | 8.98 | 3.9605 |
| | 9 | 8.46 | 4.0764 |

### Example 23: Moisture Ingress

A moisture ingress study was performed for each of the batches. The fill material was tested at the beginning, middle and end of the encapsulation process to test for moisture ingress from the capsule shell into the fill material. The fill material was removed from select capsules immediately, and after 5-, 10-, and 15-minutes hold time and tested for water content and showed that any water ingress that occurred during encapsulation was not significant. Results are summarized in Table 40.

TABLE 40
Moisture Ingress Study for 2 mg, 5 mg and 25 mg Capsules

| | Time | Av Moisture (% Water), n = 2 | | |
|---|---|---|---|---|
| Sample | (mins) | 25 mg | 5 mg | 2 mg |
| Beginning | 0 | 0.4530 | 2.0601 | 1.0275 |
| | 5 | 1.4320 | 3.4831 | 3.0461 |
| | 10 | 1.6835 | 4.7439 | 4.8145 |
| | 15 | 1.8855 | 5.8772 | 1.5458 |
| Middle | 0 | 0.5393 | 0.3962 | 1.4367 |
| | 5 | 1.8137 | 1.3670 | 2.5519 |
| | 10 | 2.7736 | 2.7075 | 4.3200 |
| | 15 | 2.4592 | 4.5432 | 6.0708 |
| End | 0 | 0.6124 | 5.1609 | 1.7996 |
| | 5 | 1.4599 | 5.5292 | 1.8779 |
| | 10 | 0.8828 | 5.4008 | 4.7468 |
| | 15 | 2.2376 | 5.2813 | 5.1932 |

### Example 24: 2 Mg & Placebo: 5 Mg & Placebo: 10 Mg, 20 Mg, 25 Mg & Placebo Capsules

Soft gelatin capsules (Softgel) for immediate release, oral administration, were prepared in 2 mg, 5 mg, 10 mg and 25 mg strengths. The 2 mg, 5 mg and 10 mg soft gelatin capsules were opaque, oval, blue colored with no print, containing a pale yellow to green opaque semisolid, sized <22 mm in largest dimension. The 25 mg soft gelatin capsules were opaque, oblong, blue colored with no print, containing a pale yellow to green opaque semisolid.

| Weight | 2 mg | 5 mg | 10 mg | 25 mg |
|---|---|---|---|---|
| Size | 3 Oval | 4 Oval | 7.5 Oval | 20 Oblong |

The content uniformity, residual solvents and elemental impurities of the capsules conformed to pharmaceutical standards (USP <905>; USP <467> Option 1; and USP <232>/<233> respectively). Any unspecified degradation products were present at <1.0% and total degradation products were <3.0%.

All the capsules containing Compound 1 have a common fill and the same gelatin composition and thickness. Table 40 presents the capsule fill components and Table 41 presents the capsule shell components. Table 42 presents the source and function of the capsule ingredients

TABLE 40
Theoretical amounts of fill ingredients per capsule (mg)

| | Active | | | | | Placebo | | |
|---|---|---|---|---|---|---|---|---|
| Ingredient | 2 mg | 5 mg | 10 mg | 20 mg | 25 mg | 2 mg | 5 mg | 25 mg |
| Compound 1 | 2.00 | 5.00 | 10.00 | 20.00 | 25.00 | 0.00 | 0.00 | 0.00 |
| Polyethylene Glycol 400 | 26.40 | 66.00 | 132.00 | 264.00 | 330.00 | 27.28 | 68.20 | 341.00 |
| PGM* | 17.60 | 44.00 | 88.00 | 176.00 | 220.00 | 18.48 | 46.20 | 231.00 |
| Vitamin E TPGS | 41.98 | 104.96 | 209.91 | 419.82 | 524.78 | 42.22 | 105.56 | 527.78 |
| Butylated Hydroxytoluene | 0.02 | 0.04 | 0.09 | 0.18 | 0.22 | 0.02 | 0.04 | 0.22 |
| TOTAL | 88.00 | 220.00 | 440.00 | 880.00 | 1100.00 | 88.00 | 220.00 | 1100.00 |

TABLE 41
Calculated amounts of shell ingredients per softgel on dry basis (mg)

| Ingredient | 2 mg | 5 mg | 10 mg | 25 mg |
|---|---|---|---|---|
| Gelatin, (Type 195) | 92.436 | 92.847 | 126.124 | 292.510 |
| Sorbitol Special-Glycerin Blend | 64.045 | 64.330 | 87.386 | 202.667 |
| Titanium dioxide | 0.440 | 0.442 | 0.601 | 1.393 |
| FD&C Blue #1 | 0.055 | 0.055 | 0.075 | 0.174 |
| Iron Oxide, Red | 0.017 | 0.017 | 0.023 | 0.052 |

The following processing aids (typically used in soft gelatin encapsulation) were also employed: fractionated coconut oil (triglycerides, medium chain) lubricates the gelatin ribbon to prevent sticking to tooling; and unbleached soy lecithin minimizes capsules sticking to each other during processing.

TABLE 42
Source and function of the capsule ingredients

| Ingredient | Source | Function |
|---|---|---|
| Fill | | |
| Compound 1 | | Active |
| Polyethylene Glycol 400 | Dow | Solubilizer |
| Propylene Glycol | Gattefosse | Surfactant |
| Monolaurate* | | (water insoluble) |
| Vitamin E TPGS | Isochem | Surfactant |
| | | (water dispersible) |
| Butylated Hydroxytoluene | Millipore | Antioxidant |
| | Sigma | |
| Shell | | |
| Gelatin, (Type 195) | Gelita USA | Shell polymer/ matrix |
| Sorbitol Special-Glycerin | SPI pharma | Plasticizer |
| Blend | | |
| Titanium dioxide | Sensient colors | Coloring agent |
| FD&C Blue #1 | Sensient colors | Coloring agent |
| Iron Oxide, Red | Sensient colors | Coloring agent |
| Processing Aid | | |
| Fractionated coconut oil | Abitec | Lubricant |
| Lecithin | ADM | Lubricant |

*PGM = Propylene Glycol Monolaurate (Lauroglycol 90)

Encapsulation of the 2 mg capsules resulted in suboptimal seals. Leaks were observed on stability at accelerated conditions, at the 3-month timepoint at both 30° C./65% RH and 40° C./75% RH conditions and were determined to be caused by the die used for encapsulation.

A tooling study was performed to establish a suitable die set. All pockets were tested for each die and it was confirmed that G2VD (full die, 8 pockets across and 200 pockets total) resulted in sub optimal seals. The G3VAL die produced the best seals of those tested.

A study was performed to determine the appropriate tooling for the 10 mg capsules, with die set G7.5VK producing optimal results. In-Process Checks (IPC) for fill weight, shell weight and seal thickness were performed at the beginning and end of encapsulation (Table 43).

TABLE 43
In process checks for 10 mg Capsules

| Sample | Acceptance Criteria | Result (n = 12) |
|---|---|---|
| Fill weight | Target: 0.440 g | Average: 0.442 g |
| | Range: 0.427-0.453 g | Range: 0.434-0.447 g |
| Shell weight | Target: 0.307 g | Average: 0.316 g |
| | Range: 0.282-0.331 g | Range: 0.304-0.330 g |
| Seal thickness | Action Limit: <0.012 inch | Leading Seal Average: 0.020 inch |
| | Minimum: 0.010 inch | Range: 0.018-0.024 inch |
| | | Trailing Seal Average: 0.015 inch |
| | | Range: 0.012-0.019 inch |

Bricking occurs when softgels, usually packaged in bottles, stick together and form a “brick”. The severity of the bricking can range from slight, where light tapping of the bottles can dislodge the brick, to severe, where the softgels will not separate. Bricking is typically observed during stability assessments at accelerated conditions, most commonly in hydrophilic fill formulations. Bricking was observed for the 2 mg, 5 mg and 25 mg capsules under accelerated conditions. A drying study was performed to determine the optimal drying time to achieve equilibrium water content of the fill material. The capsules were tested for hardness, fill moisture and water activity until an equilibrium was reached before completing drying to ensure as much water was removed as possible.

### Example 25: Finished Product Testing

The finished capsules were analyzed, and the results are shown in Table 44.

TABLE 44
Finished Product Testing

| | | Result | | |
|---|---|---|---|---|
| Test | | 25 mg | 5 mg | 2 mg |
| HPLC | | 104.3% | 102.1% | 96.9% |
| Hardness | | Av: 6.7N | Av: 7.8N | Av: 8.4N |
| Fill Moisture | [USP <921>] | 3.58% | 3.51% | 3.36% |
| Content | Uniformity | Av = 6.3 | Av = 8.6 | Av = 6.1 |
| Disintegration | [USP <701>] | Max: 14 mins | Max: 14 mins | Max: 12 mins |
| | | Av: 13 mins | Av: 13 mins | Av: 12 mins |
| Related | RRT 0.917 | <LOQ | <LOQ | <LOQ |
| Substances | RRT 0.935 | 0.93% | 0.95% | 0.94% |
| | RRT 0.985 | 1.7% | 1.8% | 1.9% |
| | RRT 1.127 | 0.25% | 0.25% | 0.25% |
| | RRT 1.168 | <LOQ | <LOQ | <LOQ |
| | Total: | 2.9% | 3.0% | 3.0% |

### Example 26: Phase I Clinical Trail

### A Phase 1 Dose Escalation Study in Healthy Subjects to Evaluate:

Safety, Tolerability, Pharmacokinetics, and Pharmacodynamics of Compound 1,
Effect of Food on Compound 1 Pharmacokinetics,
Effect of Formulation on Compound 1 Pharmacokinetics, and
CYP3A Mediated Drug-Drug Interactions with Compound 1.

The study investigates the administration of Compound 1 to healthy human subjects, to evaluate the safety, tolerability and PK of single and multiple-escalating doses. Additional objectives include assessing the effect of formulation on PK, the effect of food on PK, the effect of a CYP3A inhibitor on PK and the effect of Compound 1 on CYP3A activity. PK parameters including AUC, AUC, Cₘₐₓ, and Tₘₐₓ are determined. An additional exploratory objective evaluates pharmacodynamics, as the time-course of BTK inhibition. The study is conducted with up to 144 healthy male and female subjects, aged 18-55 years, with a body weight over 48 kg, BMI 18.5-30.0 kg/m², with no clinically significant abnormalities.

The study consisted of 3 parts (Part A-C) as described below.

### Part a (Dose Escalation, Single and Multiple Ascending Doses)

Part A is a randomized, double-blind, placebo-controlled, single- and -multiple-dose study with staggered dose escalation, administered in the fed state (standard moderate-fat meal). Part A consists of 9 cohorts (up to 7 single-ascending dose [SAD] cohorts [Cohorts 1-6 & 14], and up to 3 multiple ascending dose [MAD] cohorts [Cohorts 7-9]). Subjects are randomized in a 3:1 ratio per cohort to receive either: Compound 1 Formulation A (N=6 for Cohorts 1-4 and N=9 for Cohorts 5-9 & 14); or placebo (PBO; N=2 for Cohorts 1-4 and N=3 for Cohorts 5-9 & 14). Formulation A used in the clinical trial protocol of this example is equivalent to Formulation A7 of Example 18.

### Single-Ascending Dose Cohorts: Cohorts 1-6

Cohorts 1-4: Day 1, all subjects receive a single dose of Formulation A Compound 1 or placebo.
Cohorts 5, 6 & 14: Day −1, all subjects receive placebo (establish baseline ECG assessments). Day 1, subjects receive a single oral dose of Compound 1 or placebo.

| Cohort | Day 1 | Dose | State |
|---|---|---|---|
| 1 | Cmpd 1 (5 mg) (N = 6) or placebo (N = 2) | Single | Fed |
| 2 | Cmpd 1 (15 mg) (N = 6) or placebo (N = 2) | Single | Fed |
| 3 | Cmpd 1 (45 mg) (N = 6) or placebo (N = 2) | Single | Fed |
| 4 | Cmpd 1 (5 mg + 10 mg placebo) (N = 6) or placebo | Single | Fed |
| | (N = 2) | | |
| 5 | Cmpd 1 (100 mg) (N = 9) or placebo (N = 3) | Single | Fed |
| 6 | Cmpd 1 (200 mg) (N = 9) or placebo (N = 3) | Single | Fed |
| 14 | Cmpd 1 (300 mg) (N = 9) or placebo (N = 3) | Single | Fed |

### Multiple-Ascending Dose Cohorts: (Cohorts 7-9)

Cohorts 7 and 8: Day 1, all subjects begin once-daily multiple dose administration of Formulation A Compound 1 or placebo for 10 days.
Cohort 9: Day −1, all subjects receive placebo.
   Day 1, all subjects begin once-daily multiple dose administration of
   Compound 1 or placebo for 10 days.

| Cohort | Days 1-10 | State |
|---|---|---|
| 7 | Cmpd 1 (15 mg) (N = 9) or placebo (N = 3) | Fed |
| 8 | Cmpd 1 (50 mg) (N = 9) or placebo (N = 3) | Fed |
| 9 | Cmpd 1 (dose TBD) (N = 9) or placebo (N = 3) | Fed |

Initiation of a single dose cohort at a higher dose is determined upon review of safety data through 7 days post-last dose from all subjects enrolled in the previous dosing cohort. In the absence of dose-limiting toxicity, the cohort of the next higher dose commences.

### Part B

Based on safety data from Part A, Part B is initiated with doses at or below the highest dose evaluated in Part A.

### Formulation Effect

Cohort 10 is a randomized, 2-treatment, 2-period, 2-sequence, single-dose level formulation effect evaluation.
In Treatment Sequence AB, subjects (N=5) receive Compound 1 Formulation A in the fed state on Day 1, followed by Formulation B administered in the fed state on Day 8.
In Treatment Sequence BA, subjects (N=5) receive Compound 1 Formulation B in the fed state on Day 1, followed by Formulation A administered in the fed state on Day 8.
Days 2-7 are washout days.

| Cohort | Treatment | N | Day 1 | Day 8 |
|---|---|---|---|---|
| 10 | AB | 5 | Cmpd 1 Form A | Cmpd 1 Form B |
| | BA | 5 | Cmpd 1 Form B | Cmpd 1 Form A |

### Food Effect

Cohort 11 is a randomized, 2-treatment, 2-period, 2-sequence, single-dose level food effect evaluation. The formulation used in Cohort 11 is Formulation A. In Treatment Sequence FH, subjects receive Compound 1 in the fasted state on Day 1, and a second dose in the fed state (high-fat, high-calorie meal) on Day 8. In Treatment Sequence HF, subjects receive Compound 1 in the fed state (high-fat, high-calorie meal) on Day 1 and a second dose in the fasted state on Day 8. Days 2-7 are washout days.

### Overview of Treatment Periods for Part B (Cohorts 10 and 11)

| Cohort | Treatment | N | Day 1 | Day 8 |
|---|---|---|---|---|
| 11 | FH | 5 | Cmpd 1 15 mg, | Cmpd 1 15 mg, fed |
| | | | fasted | (high-fat meal) |
| | HF | 5 | Cmpd 1 15 mg, fed | Cmpd 1 15 mg, |
| | | | (high-fat meal) | fasted |

Part C (Drug-Drug Interaction with CYP3A)

In Part C, fed administration consists of a standard moderate-fat meal. The formulation utilized is Formulation A. The dose for Part C is based on the safety data from Part A.

Cohort 12 is an open-label, fixed-sequence, multiple-dose DDI study with itraconazole (ITZ). Subjects receive a single oral dose of Compound 1 administered in the fasted state on Day 1. Beginning on Day 3 and continuing through to Day 6, subjects receive itraconazole 200 mg once daily (QD) in a fasted state. On Day 5, subjects receive Compound 1 administered in the fasted state, 1 hour after administration of itraconazole.

| Cohort | Day 1 | Day 2 | Days 3-4 | Day 5 | Day 6 |
|---|---|---|---|---|---|
| 12 | Cmpd 1 | Wash | ITZ | Cmpd 1 | ITZ (200 mg) |
| (N = 9) | 2 mg | out | (200 mg) | 2 mg + | |
| | | | | ITZ (200 mg) | |

Cohort 13 is an open-label, fixed sequence, multiple-dose DDI study with midazolam (MDZ). Subjects receive an oral dose of midazolam (2 mg) in the fed state on Day 1. Beginning on Day 3 and continuing through Day 12, subjects receive Compound 1 twice daily (BID) in the fed state. On Days 3 and 11, subjects receive a single oral dose of midazolam (2 mg) administered simultaneously with the morning dose of Compound 1, in the fed state.

| Cohort | Day 1 | Day 2 | Day 3 | Days 4-10 | Day 11 | Day 12 |
|---|---|---|---|---|---|---|
| 13 | Single dose | Wash | Cmpd 1 | Cmpd 1 | Cmpd 1 | Cmpd 1 |
| (N = 9) | MDZ (2 mg) | out | TBDmg, BID + | TBDmg, BID | TBDmg, BID + | TBDmg, BID |
| | | | single dose | | single dose | |
| | | | MDZ (2 mg) | | MDZ (2 mg) | |

ITZ = itraconazole; MDZ = midazolam; BID = twice daily; QD = once daily.

### Number of Subjects

| | | | |
|---|---|---|---|
| Part A (N = 104) | | | |
| Cohorts 1-4: | 8 subjects | 6 Compound | (32 total) |
| | per cohort | 1 + 2 placebo | |
| Cohorts 5-9 & 14: | 12 subjects | 9 Compound | (60 total) |
| | per cohort | 1 + 3 placebo | |
| Part B (N = 22) | | | |
| Cohort 10: | 10 subjects | all Compound 1 | |
| Cohort 11: | 12 subjects | all Compound 1 | |
| Part C (N = 18) | | | |
| Cohorts 12-13: | 9 subjects | all Compound 1 | |
| | per cohort | | |

### Study Duration

Screening: 28 days
Confinement:

| | | | |
|---|---|---|---|
| Cohorts 1-4: | 4 days, 3 nights | (Days −1 to 3) | |
| Cohorts 5, 6% 14: | | 5 days, 4 nights | (Days −2 to 3) |
| Cohorts 7 & 8: | 13 days, 12 nights | (Days −1 to 12) | |
| Cohort 9: | 14 days, 13 nights | (Days −2 to 12) | |
| Cohorts 10 & 11: | 11 days, 10 nights | (Days −1 to 10) | |
| Cohort 12: | 8 days, 7 nights | (Days −1 to 7) | |
| Cohort 13: | 14 days, 13 nights | (Days −1 to 13) | |

Follow-up Visit: ˜7 days after the final dose
Total study duration, (Screening, Confinement, and Follow-up): ˜39 to 49 days

### Formulation a and Formulation B

Compound 1 is provided as blue soft gelatin capsules for oral administration. Each capsule contains a lipid-based fill solution composed of Compound 1 dissolved in PEG 400, propylene glycol monolaurate, vitamin E polyethylene glycol succinate (TPGS), and butylated hydroxytoluene (BHT). The capsules are filled with sufficient fill solution to contain 2 mg, 5 mg or 25 mg of Compound 1. The soft gelatin capsule shell material contains gelatin (type 195), sorbitol-glycerin blend (A810), titanium dioxide, FD&C Blue #1 colorant and red iron oxide. Formulation B is evaluated based on safety and PK data from Part A.

### Example 27: Results of Clinical Trail

### Safety, Tolerability, and Pharmacokinetic Profile of Single and Multiple Ascending Doses of Compound 1 in Healthy Subjects

This first-in-human study was designed to evaluate the single- and multiple dose safety, tolerability, pharmacokinetics (PK; including CNS penetrance) and pharmacodynamics (PD), effect of food, drug interactions, and cardiac safety of Compound 1 to inform dosing and concomitant medications in clinical studies, as described in Example 25, Part A.

Methods: This is an ongoing Phase 1, double-blind, randomized, placebo-controlled, single ascending and multiple ascending dose (SAD, MAD) study in healthy subjects, with staggered dose escalations and adaptive dose selection.

Safety is assessed throughout the study and PK and PD are characterized using serial blood samples collected up to 48 hours post-dose for Compound 1 plasma concentrations, BTK target occupancy (TO) and target engagement (TE). Cerebrospinal fluid (CSF) samples are collected at 2 hours post-dose to characterize the relationship between Compound 1 plasma concentrations and CNS penetrance (CSF/Unbound plasma concentration). Compound 1 PK parameters (e.g., AUCₗₐₛₜ, AUC_{inf}, Cₘₐₓ, Tₘₐₓ, t_{1/2}) are estimated by standard noncompartmental methods.

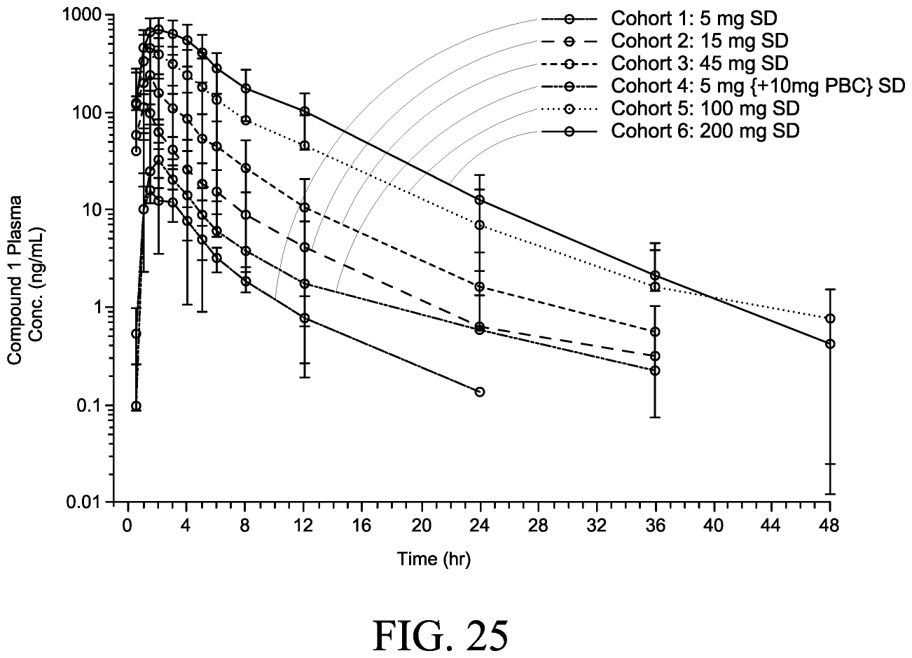

Results: Preliminary pharmacokinetic results from 3 completed SAD cohorts are presented, including 41 subjects (7 receiving 5 mg, 8 receiving 15 mg, 8 receiving 45 mg, 8 receiving 5 mg+10 mg placebo, 12 receiving 100 mg and 12 receiving 200 mg: randomized in a 3:1 ratio to receive Compound 1 or placebo) in a fed state (moderate-fat meal). A review of blinded safety data thus far indicated that study drug was generally well tolerated with no serious adverse events or early study discontinuations reported. Preliminary PK parameters are presented in Table 45 (all pharmacokinetic parameters of Table 45 are reported as Mean (% coefficient of variation), except for Tₘₐₓ and t/2, which are reported as Median (Min, Max)), and in FIG. 25 presenting the mean (SD) plasma concentration-time profiles of Compound 1.

TABLE 45
Compound 1 Parameters

| | Dose | | AUC_{0-inf} | Cₘₐₓ | Tₘₐₓ | T_{1/2} |
|---|---|---|---|---|---|---|
| Cohort | (mg) | N | (hr*ng/mL) | (ng/mL) | (hr) | (hr) |
| 1 | 5 | 5 | 62.07 | 22.5 | 2.0 | 2.77 |
| | | | (40.93%) | (56.16%) | (1.5, 3.0) | (2.06, 3.83) |
| 4 | 5 (+10 | 6 | 121.4 | 36.0 | 1.75 | 3.33 |
| | PBO) | | (73.4) | (45.0) | (1.5, 2.0) | (1.97, 4.97) |
| 2 | 15 | 6 | 338.6 | 124.0 | 1.0 | 3.60 |
| | | | (51.01%) | (33.9%) | (0.5, 1.5) | (2.26, 4.70) |
| 3 | 45 | 6 | 858.9 | 282.7 | 1.5 | 3.40 |
| | | | (65.7%) | (40.6%) | (1.0, 1.5) | (2.25, 4.83) |
| 5 | 100 | 9 | 2292 | 555.2 | 1.5 | 4.29 |
| | | | (68.3%) | (51.1%) | (1.0, 4.0) | (2.40, 5.71) |
| 6 | 200 | 9 | 4475 | 814 | 3.0 | 4.50 |
| | | | (38.3%) | (21.5) | (1.0, 4.0) | (2.89, 6.24) |

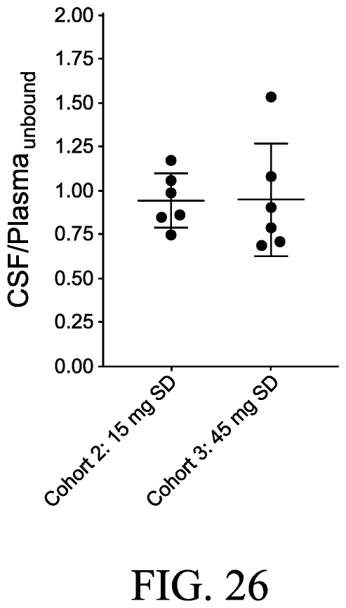

Compound 1 was rapidly absorbed following oral administration with mean plasma Tₘₐₓ ranging from 1-3 hours and exhibited a terminal half-life of approximately 3-4 hours. Single-dose Compound 1 exposure (AUC_{inf}) increased in a greater than dose-proportional manner from 5 to 15 mg and near dose-proportional from 15 to 200 mg. Mean Compound 1 CSF:unbound plasma concentration ratios approximated 1 indicating unimpeded access of unbound drug in plasma into the CNS (see, e.g., Table 46, reported as Mean (% coefficient of variation), and FIG. 26 showing CSF to unbound plasma ratio by dose). The observed Compound 1 plasma exposure, CNS penetrance, and preliminary TO and TE PD data were consistent with that of a translational PK/PD model which indicated that low doses of Compound 1 would result in high levels of BTK inhibition in blood and CNS.

TABLE 46
CSF/Plasma Concentrations

| | | | | Unbound | | |
|---|---|---|---|---|---|---|
| | | | Plasma | Plasma | CSF | |
| | | | Conc | Conc | Conc | |
| | Dose | | @2 hr | @2 hr | (2 hr | CSF/Unbound |
| Cohort | (mg) | N | (ng/mL) | (ng/mL) | (ng/mL) | Plasma |
| 2 | 15 | 6 | 63.2 | 0.83 | 0.90 | 0.95 |
| | | | (34.0%) | (34.0%) | (42.8%) | (16.3%) |
| 3 | 45 | 6 | 159 | 2.33 | 2.07 | 0.95 |
| | | | (52.6%) | (52.8%) | (51.8%) | (33.8%) |

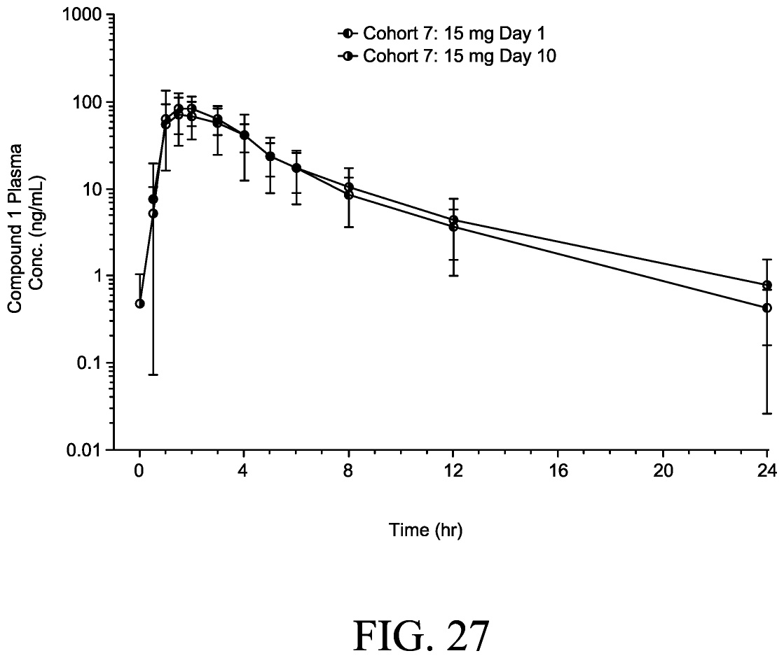

Results: Preliminary pharmacokinetic results from 1 completed MAD cohort are presented, including 12 subjects receiving 15 mg once daily for 10 days randomized in a 3:1 ratio to receive Compound 1 or placebo) in a fed state (moderate-fat meal). A review of blinded safety data thus far indicated that study drug was generally well tolerated with no serious adverse events or early study discontinuations reported. Preliminary PK parameters are presented in Table 47 (all pharmacokinetic parameters of Table 46 are reported as Mean (% coefficient of variation), except for Tₘₐₓ and t_{1/2}, which are reported as Median (Min, Max), and in FIG. 27 showing single and multiple dose plasma profiles (Mean±SD).

TABLE 47
MAD PK Parameters

| | Dose | | | AUC_{0-inf or tau} | Cₘₐₓ | Tₘₐₓ | T_{1/2} |
|---|---|---|---|---|---|---|---|
| Cohort | (mg) | Day | N | (hr*ng/mL) | (ng/mL) | (br) | (hr) |
| 7 | 15 | 1 | 9 | 312 | 85.8 | 1.5 | 3.19 |
| | | | | (45.2%) | (37.5%) | (1, 4) | (2.40, 4.24) |
| | | 10 | 9 | 353 | 101.6 | 1.5 | 3.37 |
| | | | | (40.9%) | (54.6) | (1, 2) | (1.84, 5.37) |

Compound 1 was rapidly absorbed following oral administration with mean plasma Tₘₐₓ of 1.5 hours and exhibited a terminal half-life of approximately 3 hours. Minimal to no accumulation of Compound 1 was observed upon once-daily dosing.

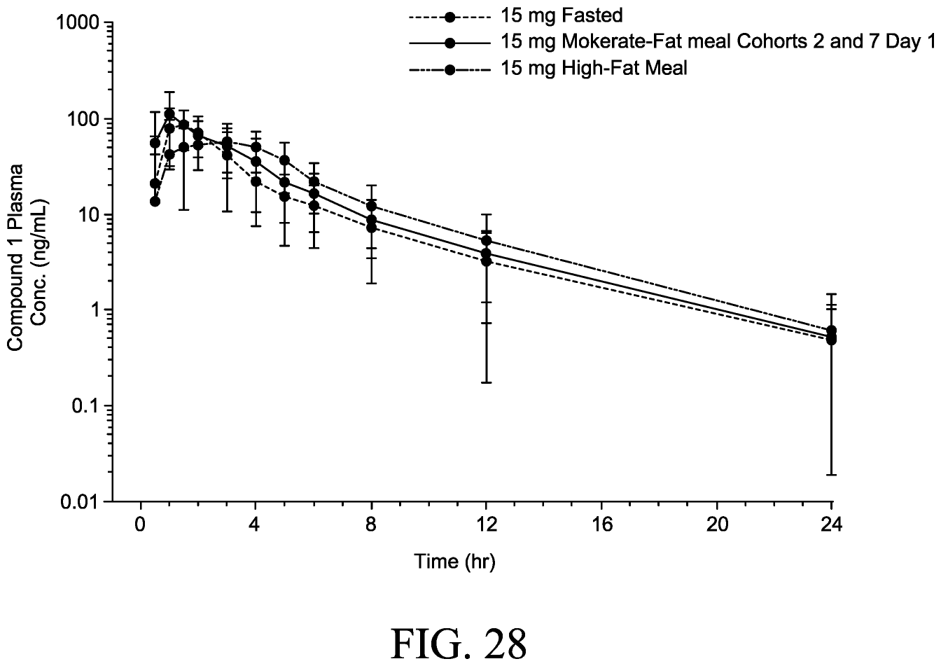

Results: Preliminary pharmacokinetic results evaluating the effect of food on Compound 1 PK are presented, including 12 subjects from Cohort 11, and 15 subjects from Cohorts 2 and 15 receiving single doses of 15 mg under fasting conditions, or in a fed state (with a moderate-fat meal or with a high-fat high-calorie meal). Preliminary PK parameters are presented in Table 48 (all pharmacokinetic parameters of Table 47 are reported as Mean (% coefficient of variation), except for Tₘₐₓ and t/2, which are reported as Median (Min, Max)), and in FIG. 28.

TABLE 48
Single-Dose PK Parameters

| | Dose | | | AUC_{0-inf} | Cₘₐₓ | Tₘₐₓ | T_{1/2} |
|---|---|---|---|---|---|---|---|
| Cohort | (mg) | Treatment | N | (hr*ng/mL) | (ng/mL) | (hr) | (hr) |
| 11 | 15 | Fasted | 11 | 312.3 | 137.2 | 1.0 | 2.89 |
| | | | | (48.9%) | (42.2%) | (1.0, 2.0) | (1.90, 5.02) |
| 2 and 7 | 15 | Moderate- | 15 | 321.5 | 101.1 | 1.5 | 2.90 |
| | | Fat | | (46.1%) | (39.5%) | (1.0, 4.0) | (1.98, 4.90) |
| 11 | 15 | High-Fat | 12 | 356.1 | 85.5 | 2.5 | 2.99 |
| | | | | (37.3%) | (41.6%) | (1.0, 5.0) | (1.98, 4.90) |

| Moderate-Fat/Fasted | 107.9 | 74.1 | N/A |
|---|---|---|---|
| % GMR (90% CD) - unpaired | (76.7, 153.0) | (54.7, 100.5) | |
| High-Fat/Fasted | 120.3 | 68.0 | |
| % GMR (90% CD) -paired | (81.3, 178.0) | (50.66, 91.2) | |

Compound 1 was rapidly absorbed following oral administration with mean plasma Tₘₐₓ of 1.0-2.5 hours and exhibited a terminal half-life of approximately 3 hours. Food (moderate fat, or high-fat high calorie meal) had no clinically significant effect on the PK of Compound 1.

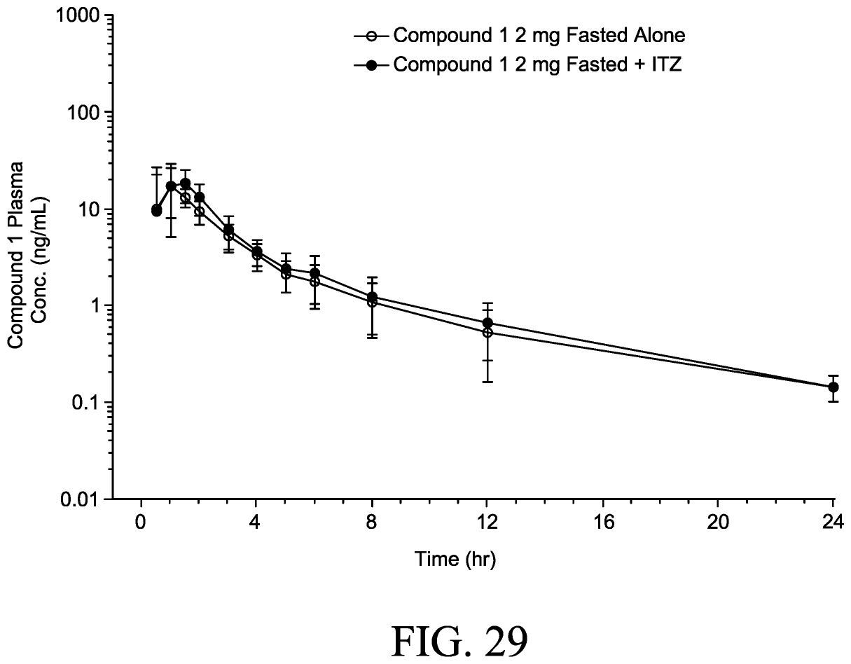

Results. Preliminary pharmacokinetic results evaluating the drug-drug interaction effect of a strong CYP3A inhibitor (itraconazole) on Compound 1 PK are presented, including 9 subjects from Cohort 12 receiving single doses of 2 mg Compound 1 under fasting conditions alone or in combination with once daily itraconazole 200 mg. Preliminary PK parameters are presented in Table 49 (all pharmacokinetic parameters of Table 49 are reported as Mean (% coefficient of variation), except for Tₘₐₓ and t_{1/2}, which are reported as Median (Min, Max)), and in FIG. 29 presenting the mean (SD) plasma concentration-time profiles of Compound 1.

TABLE 49
MAD PK Parameters

| | Dose | | | AUC_{0-inf} | Cₘₐₓ | Tₘₐₓ | T_{1/2} |
|---|---|---|---|---|---|---|---|
| Cohort | (mg) | Day | N | (hr*ng/mL) | (ng/mL) | (hr) | (hr) |
| 12 | 2 | 1 | 9 | 46.7 | 21.2 | 1.0 | 3.13 |
| | | (Alone) | | (30.3%) | (40.8) | (0.5, 2.0) | (2.01, 5.03) |
| | | 5 | 9 | 54.4 | 26.3 | 1.5 | 3.14 |
| | | (+ITZ) | | (34.9%) | (43.5) | (0.5, 2.0) | (1.54, 4.57) |

| % GMR (90% CI) →ITZ/Alone | 115% | 123% |
|---|---|---|
| | (105, 126) | (97.8, 156) |

Compound 1 was rapidly absorbed following oral administration with mean plasma Tₘₐₓ of 1.0-1.5 hours and exhibited a terminal half-life of approximately 3 hours. Coadministration of itraconazole minimally increased Compound 1 AUC_{inf} (15%) and Cₘₐₓ (23%). Coadministration of a strong CYP3A inhibitor with Compound 1 had no clinically significant effect on the PK of Compound 1.

Conclusions: Data from the first 6 SAD cohorts and the first MAD cohort in healthy subjects indicate that low doses of Compound 1 provided plasma exposure and CSF penetrance adequate to drive desired BTK inhibition in the CNS. Neither food nor coadministration of strong CYP3A inhibitors have a clinically significant effect of the PK of Compound 1. Blinded safety data indicate that single-dose study drug administration was well tolerated across the dose range.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

All publications, patents, and patent applications mentioned in this specification are herein incorporated by reference to the same extent as if each individual publication, patent, or patent application was specifically and individually indicated to be incorporated by reference.

This application claims the benefit of priority to U.S. Provisional Application No. 63/257,509, filed Oct. 19, 2021, and U.S. Provisional Application No. 63/393,163, filed Jul. 28, 2022, which applications are hereby incorporated by reference in their entirety.

1. A solid crystalline form of (S)-1-(1-acryloylpiperidin-3-yl)-2-fluoro-5,6,7,8,9,10-hexahydrocyclohepta[b]indole-4-carboxamide.

2. The solid crystalline form of claim 1, wherein the crystalline form is Form I.

3. The crystalline form of claim 2, characterized by a XRPD pattern having peaks at 9.2011±0.2, 13.9620±0.2, and 16.1506±0.2 degrees 2-theta.

4. The crystalline form of claim 2, characterized by a XRPD pattern having peaks at 20.4516±0.2, 8.0416±0.2, and 13.3485±0.2 degrees 2-theta.

5. The crystalline form of claim 3, further characterized by an XRPD pattern substantially as shown in FIG. 1.

6. The solid crystalline form of claim 2, wherein the crystalline form is substantially pure Form I.

7. The solid crystalline form of claim 1, wherein the crystalline form is Form II.

8. The crystalline form of claim 7, characterized by a XRPD pattern having peaks at 4.2759±0.2, 8.5794±0.2, and 24.2411±0.2 degrees 2-theta.

9. The crystalline form of claim 7, characterized by a XRPD pattern having peaks at 20.98±0.2, 12.07±0.2, 15.78±0.2, and 24.26±0.20.2 degrees 2-theta.

10. The solid crystalline form of any one of claims 7-9, further characterized by an XRPD pattern substantially as shown in FIG. 3.

11. The solid crystalline form of claim 7, wherein the crystalline form is substantially pure Form II.

12. The solid crystalline form of claim 1, wherein the crystalline form is Form III.

13. The crystalline form of claim 12, characterized by a XRPD pattern having peaks at 10.2543±0.2, 13.5006±0.2, and 13.9691±0.2 degrees 2-theta.

14. The crystalline form of claim 12, characterized by a XRPD pattern having peaks at 22.22±0.2, 19.27±0.2, 20.81±0.2, and 8.70±0.2 degrees 2-theta.

15. The solid crystalline form of any one of claims 12-14, further characterized by an XRPD pattern substantially as shown in FIG. 6.

16. The solid crystalline form of claim 12, wherein the crystalline form is substantially pure Form III.

17. The solid crystalline form of claim 1, wherein the crystalline form is Form IV.

18. The crystalline form of claim 17, characterized by a XRPD pattern having peaks at 8.6027±0.2, 11.9598±0.2, 13.9360±0.2, 21.5845±0.2, and 25.4090±0.2 degrees 2-theta.

19. The crystalline form of claim 17, characterized by a XRPD pattern having peaks at 19.7438±0.2, 8.3694±0.2, and 18.8538±0.2 degrees 2-theta.

20. The solid crystalline form of any one of claims 17-19, further characterized by an XRPD pattern substantially as shown in FIG. 10.

21. The solid crystalline form of claim 17, wherein the crystalline form is substantially pure Form IV.

22. The solid crystalline form of claim 1, wherein the crystalline form is Form V.

23. The crystalline form of claim 22, characterized by a XRPD pattern having peaks at 6.4014±0.2, 9.1908±0.2, 14.8143±0.2, 17.5539±0.2, 21.5891±0.2, 23.9883±0.2, and 25.5807±0.2 degrees 2-theta.

24. The crystalline form of claim 22, characterized by a XRPD pattern having peaks at 8.49±0.2, 6.11±0.2, 20.95±0.2, and 21.17±0.2 degrees 2-theta.

25. The solid crystalline form of any one of claims 22-24, further characterized by an XRPD pattern substantially as shown in FIG. 12.

26. The solid crystalline form of claim 22, wherein the crystalline form is substantially pure Form V.

27. The solid crystalline form of claim 1, wherein the crystalline form is Form VI.

28. The crystalline form of claim 27, characterized by a XRPD pattern having peaks at 6.8339±0.2, 10.1404±0.2, 15.6784±0.2, 16.1217±0.2, 17.5940±0.2, 20.6765±0.2, 25.5122±0.2, and 26.7363±0.2 degrees 2-theta.

29. The crystalline form of claim 27, characterized by a XRPD pattern having peaks at 23.93±0.2, 13.05±0.2, 18.36±0.2, and 8.54±0.2 degrees 2-theta.

30. The solid crystalline form of any one of claims 27-29, further characterized by an XRPD pattern substantially as shown in FIG. 14.

31. The solid crystalline form of claim 27, wherein the crystalline form is substantially pure Form VI.

32. The solid crystalline form of claim 1, wherein the crystalline form is Form VII.

33. The crystalline form of claim 32, characterized by a XRPD pattern having peaks at 6.727, 8.4799, 9.4854, 12.0161, 17.1901, 18.8407, 19.0691, 19.7285 and 20.2268±0.2 degrees 2-theta.

34. The solid crystalline form of any one of claims 32-33, further characterized by an XRPD pattern substantially as shown in FIG. 15.

35. The solid crystalline form of claim 32, wherein the crystalline form is substantially pure Form VII.

36. A pharmaceutical composition comprising the solid crystalline form of any one of claims 1-35.

37. The pharmaceutical composition of claim 36, comprising an additional therapeutically active compound.

38. The pharmaceutical composition of claim 36, wherein the composition is formulated for oral administration.

39. The pharmaceutical composition of claim 36, wherein the composition is in the form of a gel capsule.

40. The pharmaceutical composition of claim 36, further comprising polyethylene glycol.

41. The pharmaceutical composition of claim 36, further comprising polyethylene glycol monolaurate.

42. The pharmaceutical composition of claim 36, further comprising vitamin E.

43. The pharmaceutical composition of claim 36, further comprising butylated hydroxytoluene.

44. A pharmaceutical composition comprising 1-25 mg (S)-1-(1-acryloylpiperidin-3-yl)-2-fluoro-5,6,7,8,9,10-hexahydrocyclohepta[b]indole-4-carboxamide.

45. The pharmaceutical composition of claim 44, comprising 2 mg, or 5 mg, or 10 mg, or 20 mg, or 25 mg of (S)-1-(1-acryloylpiperidin-3-yl)-2-fluoro-5,6,7,8,9,10-hexahydrocyclohepta[b]indole-4-carboxamide.

46. A pharmaceutical composition comprising:
1-25 mg (5)-1-(1-acryloylpiperidin-3-yl)-2-fluoro-5,6,7,8,9,10-hexahydrocyclo-hepta[b]indole-4-carboxamide;
polyethylene glycol;
propylene glycol monolaurate;
vitamin E; and
butylated hydroxytoluene.

47. A method for treating a disease or condition modulated by kinase inhibition, comprising administering to a subject in need thereof an effective amount of the solid crystalline form of any one of claims 1-35, or the pharmaceutical composition of any one of claims 36-46.

48. The method of claim 47, wherein the kinase is a tyrosine kinase.

49. The method of claim 48, wherein the tyrosine kinase is Bruton's tyrosine kinase (BTK).

50. The method of claim 47, wherein the disease or condition is cancer.
